(19) United States
(12) Patent Application Publication
(10) Pub. No.: US 2026/0178916 A1
(43) Pub. Date: Jun. 25, 2026

(54) PREDICTING JOINT THREE-DIMENSIONAL (3D) STRUCTURES OF PROTEINS AND LIGANDS BY COFOLDING

(71) Applicant: Isomorphic Labs Limited, London (GB)

(72) Inventors: Maxwell Elliot Jaderberg, London (GB); Chia-Chun Hung, London (GB); Victor Constant Bapst, London (GB); Jack Tavis Dunger, London (GB); David Reiman, Santa Cruz, CA (US); Zachary Wu, Milpitas, CA (US)

(21) Appl. No.: 19/537,748

(22) Filed: Feb. 12, 2026

Related U.S. Application Data
(63) Continuation of application No. PCT/EP2024/080580, filed on Oct. 29, 2024.
(60) Provisional application No. 63/546,444, filed on Oct. 30, 2023.
(60) Provisional application No. 63/611,638, filed on Dec. 18, 2023.

(51) Int. Cl. G06N 3/084

(52) U.S. Cl. CPC G06N 3/084

(57) ABSTRACT

Methods, systems, and apparatus, including computer programs encoded on a computer storage medium, for generating a predicted joint 3D structure of a protein and one or more ligands. In one aspect, a method comprises: obtaining a network input that characterizes the protein and the one or more ligands; processing the network input using an embedding neural network to generate a protein-ligand embedding of the protein and the one or more ligands; and generating, using a generative model and while the generative model is conditioned on the protein-ligand embedding, the predicted joint three-dimensional (3D) structure of the protein and the one or more ligands.

### CROSS-REFERENCE TO RELATED APPLICATION

This application is a continuation application of and claim priority to International Application No. PCT/EP2024/080580, filed on Oct. 29, 2024, which claims priority to U.S. Provisional Application No. 63/546,444, filed on Oct. 30, 2023, and to U.S. Provisional Application No. 63/611,638, filed on Dec. 18, 2023. The disclosure of the prior applications are considered part of and are incorporated by reference in the disclosure of this application.

### BACKGROUND

This specification relates to predicting a joint three-dimensional (3D) structure of a protein and one or more ligands.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a system implemented as computer programs on one or more computers in one or more locations that can predict a joint 3D structure of a protein and one or more ligands.

A “protein” can be understood to refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

A “ligand” can refer to a molecule or compound that binds to a target molecule, e.g., a protein. Ligands can include, e.g., small organic molecules, complex organic molecules, proteins, biomolecules, and so forth.

A “multiple sequence alignment” (MSA) for an amino acid chain in a protein specifies a sequence alignment of the amino acid chain with multiple additional amino acid chains, e.g., from other proteins, e.g., homologous proteins. More specifically, the MSA can define a correspondence between the positions in the amino acid chain and corresponding positions in multiple additional amino acid chains. A MSA for an amino acid chain can be generated, e.g., by processing a database of amino acid chains using any appropriate computational sequence alignment technique, e.g., progressive alignment construction. The amino acid chains in the MSA can be understood as having an evolutionary relationship, e.g., where each amino acid chain in the MSA may share a common ancestor. The correlations between the amino acids in the amino acid chains in a MSA for an amino acid chain can encode information that is relevant to predicting the structure of the amino acid chain.

A “binding pocket” on a protein can refer to a specific three-dimensional cavity or crevice within the structure of the protein where a ligand can bind to the protein. The binding pocket can, in some cases, be understood as a “lock” that fits the shape and chemical properties of ligands that act as “keys” for the lock. In other cases, the ligand may initially not fit perfectly into the binding pocket, e.g., due to structural differences or slight mismatches in shape or chemical groups, but conformational changes during binding can cause the interaction between the ligand and the binding pocket to become more complementary and specific, e.g., as in induced-fit binding. Examples of binding pockets include, e.g., orthosteric binding pockets, allosteric binding pockets, and cryptic binding pockets.

A first neural network can be referred to as a “subnetwork” of a second neural network if the first neural network is included in the second neural network.

A “block” (e.g., a “self-attention block”) in a neural network can refer to a group of one or more neural network layers in the neural network.

An “embedding” of an entity (e.g., an atom, or a ligand, or a protein) can refer to a representation of the entity as an ordered collection of numerical values, e.g., a vector, matrix, or other tensor of numerical values.

“Conditioning” a model (e.g., a generative model) or a neural network (e.g., a denoising neural network) or an operation (e.g., a self-attention operation) on conditioning data (e.g., an embedding representing a protein and one or more ligands) can refer to providing the conditioning data as an input (e.g., a side input) to the model, neural network, or operation, such that outputs generated by the model, neural network, or operation are influenced by (depend on) the conditioning data.

A “binding affinity” of a ligand for a protein refers to the strength or degree of attraction between the ligand and the protein when they interact to form a complex. Binding affinities can be determined experimentally by, for example, measuring equilibrium constants or free energies for association of the ligand and protein.

A 3D spatial position of an atom can be represented by a set of coordinates in an appropriate coordinate system, e.g., a 3D Cartesian coordinate system or a spherical coordinate system.

According to a first aspect there is provided a method performed by one or more computers, the method comprising: obtaining a network input that characterizes a protein and one or more ligands; processing the network input characterizing the protein and the one or more ligands using an embedding neural network to generate a protein-ligand embedding of the protein and the one or more ligands; and generating, using a generative model and while the generative model is conditioned on the protein-ligand embedding, a predicted joint three-dimensional (3D) structure of the protein and the one or more ligands, wherein the predicted joint 3D structure of the protein and the one or more ligands defines a respective predicted three-dimensional spatial location of each atom in the protein and of each atom in each of the one or more ligands.

In some implementations, the embedding neural network comprises a protein embedding neural network and a ligand embedding neural network, and wherein processing the network input characterizing the protein and the one or more ligands using the embedding neural network to generate the protein-ligand embedding comprises: processing data characterizing the protein using the protein embedding neural network to generate a protein embedding of the protein; processing data characterizing the one or more ligands using the ligand embedding neural network to generate a ligand embedding of the one or more ligands; and processing the protein embedding and the ligand embedding to generate the protein-ligand embedding.

In some implementations, the protein embedding comprises a respective amino acid embedding of each amino acid in the protein; wherein the ligand embedding comprises a respective atom embedding of each atom in each of the one or more ligands; and processing the protein embedding and the ligand embedding to generate the protein-ligand embedding comprises: generating data defining a 1D sequence of amino acid embeddings and atom embeddings by concatenating: (i) the amino acid embeddings of the protein embedding, and (ii) the atom embeddings of the ligand embedding; wherein the protein-ligand embedding is derived from the 1D sequence of amino acid embeddings and atom embeddings.

In some implementations, the processing the protein embedding and the ligand embedding to generate the protein-ligand embedding further comprises: transforming the 1D sequence of amino acid embeddings and atom embeddings into a two-dimensional (2D) array of embeddings; wherein the protein-ligand embedding is derived from the 2D array of embeddings.

In some implementations, the 2D array of embeddings comprises a plurality of atom-atom embeddings that are each derived from a respective pair of atom embeddings of the ligand embedding.

In some implementations, the 2D array of embeddings comprises a plurality of amino acid-amino acid embeddings that are each derived from a respective pair of amino acid embeddings of the protein embedding.

In some implementations, the 2D array of embeddings comprises a plurality of amino acid-atom embeddings that are each derived from: (i) a respective atom embedding of the ligand embedding, and (ii) a respective amino acid embedding of the protein embedding.

In some implementations, transforming the 1D sequence of amino acid embeddings and atom embeddings into the 2D array of embeddings comprises: applying an outer product operation to the 1D sequence of amino acid embeddings and atom embeddings; or applying a 2D concatenation operation to the 1D sequence of amino acid embeddings and atom embeddings.

In some implementations, the embedding neural network further comprises a fusion neural network; and processing the protein embedding and the ligand embedding to generate the protein-ligand embedding further comprises: processing the 2D array of embeddings using the fusion neural network to generate an updated 2D array of embeddings; wherein the updated 2D array of embeddings defines the protein-ligand embedding.

In some implementations, the fusion neural network comprises a sequence of self-attention blocks, wherein each self-attention block is configured to perform operations comprising: applying one or more self-attention operations to an input 2D array of embeddings to update the input 2D array of embeddings.

In some implementations, for one or more of the self-attention blocks, the self-attention operations comprise one or more row-wise self-attention operations.

In some implementations, for one or more of the self-attention blocks, the self-attention operations comprise one or more column-wise self-attention operations.

In some implementations, for one or more of the self-attention blocks, the self-attention operations comprise one or more triangle self-attention operations.

In some implementations, the network input comprises data defining one or more of: an amino acid sequence of the protein; a multiple sequence alignment (MSA) for the protein; a respective structure of each of one or more template proteins; a representation of a respective chemical structure of each of the one or more ligands.

In some implementations, the generative model is a generative diffusion model that comprises a denoising neural network.

In some implementations, generating, using the generative model and while the generative model is conditioned on the protein-ligand embedding, the predicted joint 3D structure of the protein and the one or more ligands comprises: generating positional data defining a respective initial position of each atom in a complex comprising the protein and the one or more ligands; denoising the positional data over a sequence of time steps using the denoising neural network and while the denoising neural network is conditioned on the protein-ligand embedding; wherein the predicted joint 3D structure of the protein and the one or more ligands is defined by the positional data after a final time step in the sequence of time steps.

In some implementations, generating positional data defining the respective initial position of each atom in the complex comprises: sampling the respective initial position of each atom in the complex from a probability distribution over 3D space.

In some implementations, denoising the positional data over the sequence of time steps using the denoising neural network and while the denoising neural network is conditioned on the protein-ligand embedding comprises, at each of one or more time steps in the sequence of time steps: receiving current positional data that defines a respective current position of each atom in the complex at the time step; generating a denoising output using the denoising neural network and while the denoising neural network is conditioned on the protein-ligand embedding; and generating positional data that defines a respective position of each atom in the complex at a next time step using the denoising output.

In some implementations, the denoising output comprises a respective predicted error in the current position of each atom in the complex at the time step.

In some implementations, generating the denoising output using the denoising neural network and while the denoising neural network is conditioned on the protein-ligand embedding comprises: generating a set of atom embeddings using an encoder block of the denoising neural network, wherein each atom embedding represents one or more atoms in the complex and is based at least in part on the respective current spatial position of the one or more atoms at the time step; processing the set of atom embeddings using an update block of the denoising neural network to generate a set of updated atom embeddings; and processing the set of updated atom embeddings to generate the denoising output.

In some implementations, the set of atom embeddings includes a respective atom embedding representing each atom included in each of the one or more ligands.

In some implementations, the set of atom embeddings includes a respective atom embedding representing each atom included in each amino acid of the protein.

In some implementations, for each amino acid in the protein, the set of atom embeddings includes a respective atom embedding that jointly represents all the atoms included in the amino acid.

In some implementations, each atom embedding in the set of atom embeddings is based on, for each of the one or more atoms represented by the atom embedding: (i) the current position of the atom at the time step, and (ii) a respective conditioning embedding for the atom that is selected from a collection of embeddings included in the protein-ligand embedding.

In some implementations, for each atom embedding that represents an atom in the complex that is included in a ligand, the conditioning embedding for the atom comprises an atom-atom embedding corresponding to the atom in the protein-ligand embedding.

In some implementations, for each atom embedding that represents an atom in the complex that is included in an amino acid of the protein, the conditioning embedding for the atom comprises an amino acid-amino acid embedding corresponding to the amino acid in the protein-ligand embedding.

In some implementations, the update block of the denoising neural network comprises a sequence of self-attention blocks; wherein each of the self-attention blocks are configured to apply one or more self-attention operations to a set of current atom embeddings to update the set of current atom embeddings; wherein each of the one or more self-attention operations are conditioned on the protein-ligand embedding.

In some implementations, applying a self-attention operation to the set of current atom embeddings to update the set of current atom embeddings comprises: generating, based on the current set of atom embeddings, a respective intermediate attention score for each pair of current atom embeddings from the set of current atom embeddings; generating, based on the protein-ligand embedding, a respective attention score bias for each pair of current atom embeddings from the set of current atom embeddings; generating a respective final attention score for each pair of current atom embeddings from the set of current atom embeddings based on the intermediate attention scores and the attention score biases; and updating the set of current atom embeddings using the final attention scores.

In some implementations, for each pair of current atom embeddings from the set of current atom embeddings, generating the attention score bias for the pair of current atom embeddings comprises: summing the intermediate attention score for the pair of current atom embeddings and the attention score bias for the pair of current atom embeddings.

In some implementations, for each pair of current atom embeddings from the set of current atom embeddings, generating the attention score bias for the pair of current atom embeddings comprises: processing a respective conditioning embedding selected from a collection of embeddings included in the protein-ligand embedding using a projection neural network to generate the attention score bias.

In some implementations, for each pair of current atom embeddings that includes: (i) a first atom embedding representing a first atom included in a ligand, and (ii) a second atom embedding representing a second atom included in a ligand, the selected conditioning embedding comprises an atom-atom embedding corresponding to the first atom and the second atom in the protein-ligand embedding.

In some implementations, for each pair of current atom embeddings that includes: (i) a first atom embedding representing a first atom included in an amino acid, and (ii) a second atom embedding representing a second atom included in a ligand, the selected conditioning embedding comprises an amino acid-atom embedding corresponding to: (i) the amino acid that includes the first atom, and (ii) the second atom, in the protein-ligand embedding.

In some implementations, for each pair of current atom embeddings that includes: (i) a first atom embedding representing a first atom included in a first amino acid, and (ii) a second atom embedding representing a second atom included in a second amino acid, the selected conditioning embedding comprises an amino acid-amino acid embedding corresponding to: (i) the first amino acid, and (ii) the second amino acid, in the protein-ligand embedding.

In some implementations, for each pair of current atom embeddings that includes: (i) a first atom embedding that jointly represents all atoms in a first amino acid, and (ii) a second atom embedding that represents all atoms in a second amino acid, the selected conditioning embedding comprises an amino acid-amino acid embedding corresponding to: (i) the first amino acid, and (ii) the second amino acid, in the protein-ligand embedding.

In some implementations, for each pair of current atom embeddings that includes: (i) a first atom embedding that jointly represents all atoms in an amino acid, and (ii) a second atom embedding that represents an atom included in a ligand, the selected conditioning embedding comprises an amino acid-atom embedding corresponding to: (i) the amino acid, and (ii) the atom, in the protein-ligand embedding.

In some implementations, the method further comprises: selecting one or more of the ligands to be physically synthesized based at least in part on the predicted joint 3D structure of the protein and the one or more ligands; and physically synthesizing the selected ligands.

In some implementations, the method further comprises: selecting the protein to be physically synthesized based at least in part on the predicted joint 3D structure of the protein and the one or more ligands; and physically synthesizing the protein.

According to another aspect there is provided a method comprising: generating, for each ligand in a collection of ligands, a respective predicted joint 3D structure of the ligand and a protein using the methods described herein; determining, for each ligand in the collection of ligands, a respective predicted binding affinity of the ligand for the protein based on the predicted joint 3D structure of the ligand and the protein; and selecting one or more ligands in the collection of ligands for physical synthesis based at least in part on the predicted binding affinities.

In some implementations, the method further comprises physically synthesizing the one or more selected ligands.

According to another aspect, there is provided a method comprising: generating, for each protein in a collection of proteins, a respective predicted joint 3D structure of the protein and a ligand using the methods described herein; determining, for each protein in the collection of proteins, a respective predicted binding affinity of the ligand for the protein based on the predicted joint 3D structure of the ligand and the protein; and selecting one or more proteins in the collection of proteins for physical synthesis based at least in part on the predicted binding affinities.

In some implementations, the method further comprises physically synthesizing the one or more selected proteins.

According to another aspect there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

According to another aspect there is provided a method of obtaining a ligand, wherein the ligand is a drug or a ligand of an industrial enzyme, the method comprising: for each of one or more candidate ligands: (a) performing the method of any one of claims 1-35 to determine a predicted structure of a complex comprising a target protein molecule and the candidate ligand; and (b) evaluating an interaction of the candidate ligand with the target protein molecule dependent on the predicted structure; and selecting one or more of the candidate ligands as the ligand dependent on a result of the evaluating.

In some implementations, the target protein molecule comprises a receptor or enzyme, and wherein the ligand is an agonist or antagonist of the receptor or enzyme.

In some implementations, the ligand is a drug, and the method comprises: performing steps (a) and (b) for each of a plurality of target protein molecules; and selecting one or more of the candidate ligands as the ligand to either i) obtain a ligand that interacts with each of the target protein molecules, or ii) obtain a ligand that interacts with only one of the target protein molecules.

In some implementations, the ligand comprises an antibody or aptamer and the target protein molecule comprises an antibody or aptamer target, in particular a virus or cancer cell protein, and wherein the antibody or aptamer binds to the antibody or aptamer target to provide a therapeutic effect.

In some implementations, the ligand is a polypeptide ligand, a polynucleoside ligand, or a polynucleotide ligand.

According to another aspect there is provided a method of obtaining a diagnostic antibody or aptamer marker of a disease, the method comprising: selecting a target protein molecule; for each of one or more candidate antibodies or aptamers: performing the method of any one of claims 1-35 to determine a predicted structure of a complex comprising the candidate antibody or aptamer and the target protein molecule; and evaluating an interaction between the candidate antibody or aptamer and the target protein molecule; and selecting one of the one or more of the candidate antibodies or aptamers as the diagnostic antibody or aptamer marker dependent on a result of the evaluating.

In some implementations, the evaluating the interaction of one of the candidate ligands comprises determining an interaction score for the candidate ligand, wherein the interaction score comprises a measure of an interaction between the candidate ligand and the target molecule.

In some implementations, the method further comprises synthesizing the ligand or diagnostic antibody or aptamer marker.

In some implementations, the method further comprises testing biological activity of the ligand or diagnostic antibody or aptamer marker in vitro and in vivo.

According to another aspect, there is provided a method of determining the structure of a molecule complex comprising a protein and one or more ligands, comprising: applying an experimental technique to a physical sample comprising the molecule complex to measure experiment signals dependent on a structure of the molecule complex; performing the method of any one of claims 1-35 to determine a predicted structure of the molecule complex; using the experiment signals and the predicted structure of the molecule complex to determine the structure of the molecule complex.

In some implementations, the experimental technique comprises one or more of: x-ray crystallography, nuclear magnetic resonance, and electron microscopy.

According to another aspect there is provided a ligand that has been synthesized by performing the methods described herein.

According to another aspect there are provided one or more non-transitory computer storage media storing ligand data defining a ligand, wherein the ligand was selected from a set of candidate ligands by performing operations comprising: generating, for each candidate ligand in a collection of candidate ligands, a respective predicted joint 3D structure of the candidate ligand and a protein using the methods described herein; determining, for each candidate ligand in the collection of candidate ligands, a respective predicted binding affinity of the candidate ligand for the protein based on the predicted joint 3D structure of the candidate ligand and the protein; and selecting one or more candidate ligands in the collection of ligands for physical synthesis based at least in part on the predicted binding affinities.

According to another aspect there is provided a protein that has been synthesized by performing the methods described herein.

According to another aspect there are provided one or more non-transitory computer storage media storing ligand data defining a protein, wherein the protein was selected from a set of proteins by performing operations comprising: generating, for each protein in a collection of proteins, a respective predicted joint 3D structure of the protein and a ligand using the methods described herein; determining, for each protein in the collection of proteins, a respective predicted binding affinity of the ligand for the protein based on the predicted joint 3D structure of the ligand and the protein; and selecting one or more proteins in the collection of proteins for physical synthesis based at least in part on the predicted binding affinities.

Particular embodiments of the subject matter described in this specification can be implemented so as to realize one or more of the following advantages.

Traditionally, molecular docking of a protein and one or more ligands involves obtaining data defining respective 3D structures of the protein and the one or more ligands, and performing a search through a space of possible poses (i.e., spatial configurations) of the protein and the one or more ligands to optimize a scoring function. The scoring function can measure, e.g., the energy of each joint conformation of the protein and the one or more ligands. Conventional molecular docking can be computationally expensive, e.g., because optimizing the scoring functions requires searching a large space of possible poses of the protein and the one or more ligands. Moreover, conventional molecular docking requires advance knowledge of the individual 3D structures of the protein and the one or more ligands, and of the binding site(s) on the protein. Further, even if the 3D structure of the protein is known, e.g., from crystallography, the 3D structure of the protein may deform through a process of protein conformational change as the ligand interacts with the protein, e.g., to bind to a binding site on the protein. However, the process of conventional molecular docking does not account for potential conformational changes of the protein as a result of interaction with the ligand which can lead to inaccurate results.

The cofolding system described in this specification addresses these issues by directly mapping from: (i) data defining a protein, e.g., the amino acid sequence of the protein and an MSA for the protein, and (ii) data defining the one or more ligands, e.g., the chemical structures of the one or more ligands, to a predicted joint 3D structure of the protein and the one or more ligands. The cofolding system does not require advance knowledge of the 3D structures of the protein or the one or more ligands, and does not require advance knowledge of the binding sites on the protein. To generate the predicted joint 3D structure of the protein and the one or more ligands, the cofolding system can generate a protein-ligand embedding that jointly embeds the protein and the one or more ligands. The system can then use the protein-ligand embedding to condition a generative model that can generate one or more predicted joint 3D structures of the protein and the one or more ligands by sampling from a distribution over a space of possible 3D structures.

The co-folding system is more computationally efficient (in particular: consumes fewer computational resources, such as memory and computing power) than conventional approaches such as molecular docking. In more detail, performing molecular docking requires optimizing a scoring function by searching a large space of possible poses of the protein and the one or more ligands. In contrast, the co-folding system directly maps the protein data and the ligand data to a predicted joint 3D structure by a single forward pass through a neural network, completely avoiding the iterative search procedure that is required to perform molecular docking.

The embedding neural network can generate the protein-ligand embedding by generating a protein embedding (that includes a respective amino acid embedding for each amino acid in the protein) and a ligand embedding (that includes a respective atom embedding for each atom in each of the one or more ligands). The embedding neural network can then generate the protein-ligand embedding, e.g., by transforming the amino acid embeddings and the atom embeddings into a two-dimensional (2D) array of embeddings that captures pairwise relationships among the amino acids of the protein and the atoms of the ligand. The embedding neural network can refine the protein-ligand embedding, e.g., by performing a sequence of self-attention operations on the 2D array of embeddings to enrich each embedding with information encoded in other embeddings. The embedding neural network thus generates the protein-ligand embedding in a manner that leverages useful inductive biases specific to the problem at hand, in particular, that pairwise relationships between ligand atoms and amino acids will encode information useful for predicting their joint structure. Further, the protein-ligand embedding is derived from information encoded at multiple levels of resolution: information characterizing the protein is expressed at the lower resolution of amino acids, while information characterizing the one or more ligands is expressed at the higher resolution of atoms. Representing the protein at the lower resolution level of amino acids (rather than at the higher resolution level of atoms) can reduce consumption of computational resources, e.g., by reducing the number of embeddings over which self-attention operations are computed as part of generating the protein-ligand embedding.

The generative model, when conditioned on the protein-ligand embedding, can generate any desired number of predicted 3D structures of the protein and the one or more ligands by repeatedly sampling from a distribution over a space of possible (joint) 3D structures. Further, the generative model can be configured through training to learn to account for protein conformational changes during binding, in contrast to conventional molecular docking approaches, which assume that the protein and the ligands maintain a static, predefined structure. That is, the generative model can be trained using training examples that each correspond to a respective complex of a protein and one or more ligands that are bound to the protein, e.g., where the one or more ligands are each bound to a respective binding site on the protein.

The cofolding system can be used for any of a variety of drug discovery and drug repurposing tasks, as will be described in detail throughout this specification.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example cofolding system.

FIG. 2 shows an example embedding neural network.

FIG. 3A is a flow diagram of an example process for processing a protein embedding of a protein and a ligand embedding of one or more ligands using a fusion neural network to generate a protein-ligand embedding that jointly represents the protein and the one or more ligands.

FIG. 3B illustrates operations performed by the cofolding system to generate the protein-ligand embedding.

FIG. 4 is a flow diagram of an example process for generating a predicted joint 3D structure of a protein and one or more ligands using a generative diffusion model that includes a denoising neural network.

FIG. 5 is a flow diagram of an example process for predicting a respective error in the current 3D spatial position of each atom in the complex using a denoising neural network conditioned on a protein-ligand embedding.

FIG. 6 is a flow diagram of an example process for updating a set of current atom embeddings using a self-attention operation that is implemented by a self-attention block of the denoising neural network and that is conditioned on a protein-ligand embedding.

FIG. 7 is a flow diagram of an example process for jointly training the embedding neural network and the generative model of the cofolding system.

FIG. 8 is a flow diagram of an example process for jointly training an embedding neural network and a generative diffusion model on a training example.

FIG. 9 illustrates an example of an output generated by the cofolding system showing a predicted 3D structure of a protein and two ligands.

Like reference numbers and designations in the various drawings indicate like elements.

### DETAILED DESCRIPTION

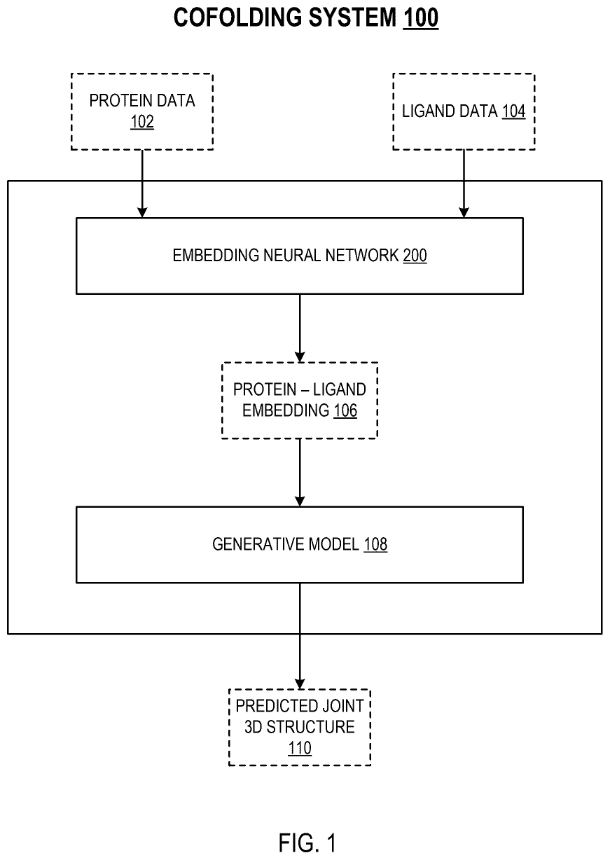

FIG. 1 shows an example cofolding system 100. The cofolding system 100 is an example of a system implemented as computer programs on one or more computers in one or more locations in which the systems, components, and techniques described below are implemented.

The cofolding system 100 is configured to process data characterizing a protein 102 (“protein data”) and data characterizing one or more ligands 104 (“ligand data”) to generate a predicted joint 3D structure 110 of a “complex” (i.e., a molecular system) that includes the protein and the one or more ligands. The predicted joint 3D structure 110 defines a respective predicted 3D spatial location of each atom in the complex, i.e., of each atom in the protein and of each atom in each of the one or more ligands. The predicted joint 3D structure can define a structure of the complex where one or more of the ligands are bound to respective binding sites on the protein.

The protein data 102 can include any appropriate data characterizing the protein, e.g., data defining one or more amino acid sequences of the protein, or data defining an MSA for the protein, or data characterizing a respective structure of each of one or more “template” proteins, or a combination thereof. A template protein can refer to a protein that is “similar” to the protein 102, e.g., such that the value of a similarity measure between the template protein and the protein 102 satisfies (e.g., exceeds) a threshold (e.g., 0.8, or 0.9, or 0.99, or any other appropriate threshold). Similarity between a first protein and a second protein can be measured using any appropriate similarity measure, e.g., a sequence identity or percent identity similarity measure between the respective amino acid sequence(s) of the first protein and the second protein. The structure of a template protein can be represented in any appropriate manner, e.g., by a contact map, or by data defining a respective 3D spatial position of each atom in the template protein. Optionally, the protein data 102 can exclude any data that directly defines the 3D structure of the protein, e.g., the 3D spatial locations of the atoms or amino acid residues in a 3D conformation of the protein.

The ligand data 104 can include any appropriate data characterizing each ligand in a set of one or more ligands. For instance, the ligand data 104 can include, for each ligand, a respective textual representation of one or more of: a chemical structure of the ligand (e.g., the arrangement of atoms and bonds in the ligand), the atom types in the ligand and their connectivity, the chirality of the bonds in the ligand, or any functional groups (e.g., hydroxyl groups, amino groups, carboxyl groups, and so forth) included in the ligand. The respective textual representation of each ligand can include, e.g., a simplified molecular-input line-entry system (SMILES) string characterizing the ligand. As another example, the ligand data 104 can include, for each ligand, a respective representation of the ligand by way of graph data representing a graph, e.g., where the nodes in the graph represent atoms in the ligand and the edges in the graph represent bonds between atoms in the ligand. Optionally, the ligand data 104 can exclude any data that directly defines the 3D structure of each ligand, e.g., the 3D spatial locations of the atoms in a 3D conformation of the ligand.

The ligand data 104 can include data characterizing any appropriate number of ligands. In some cases, the ligand data 104 characterizes one ligand. In other cases, the ligand data characterizes more than one ligand, e.g., two ligands, or three ligands, or four ligands, or five ligands.

The cofolding system 100 processes the protein data 102 and the ligand data 104 using an embedding neural network 200 and a generative model 108 to generate the predicted joint 3D structure 110 of the complex. The embedding neural network 200 and the generative model 108 are each described in more detail next (and throughout this specification).

The embedding neural network 200 is configured to process the protein data 102 and the ligand data 104 to generate a protein-ligand embedding 106 that represents the protein and the one or more ligands.

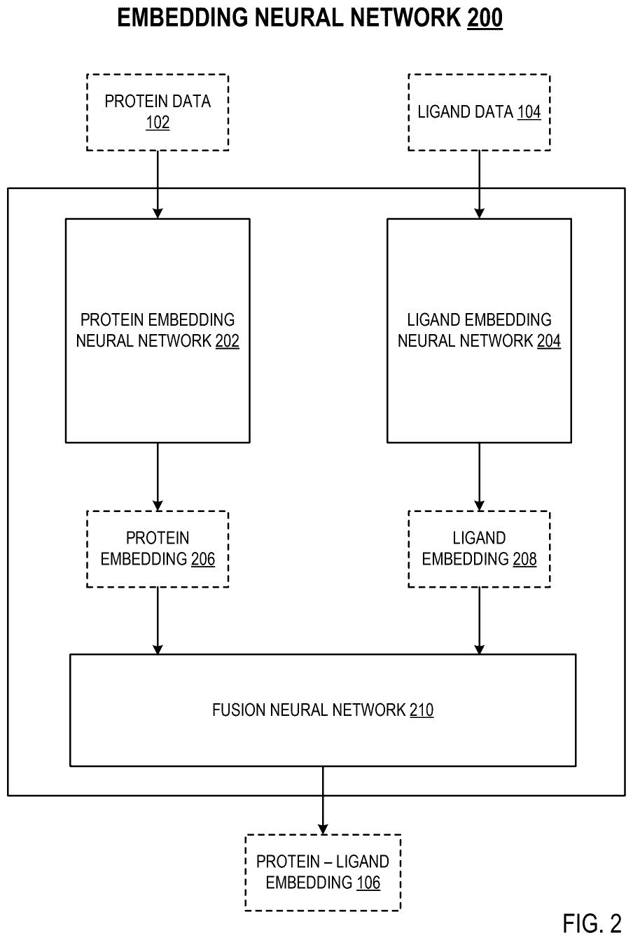

The embedding neural network 200 can have any appropriate neural network architecture that enables the embedding neural network 200 to perform its described functions. In particular, the embedding neural network 200 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers). A particular example of a possible architecture of the embedding neural network 200 is described in more detail with reference to FIG. 2.

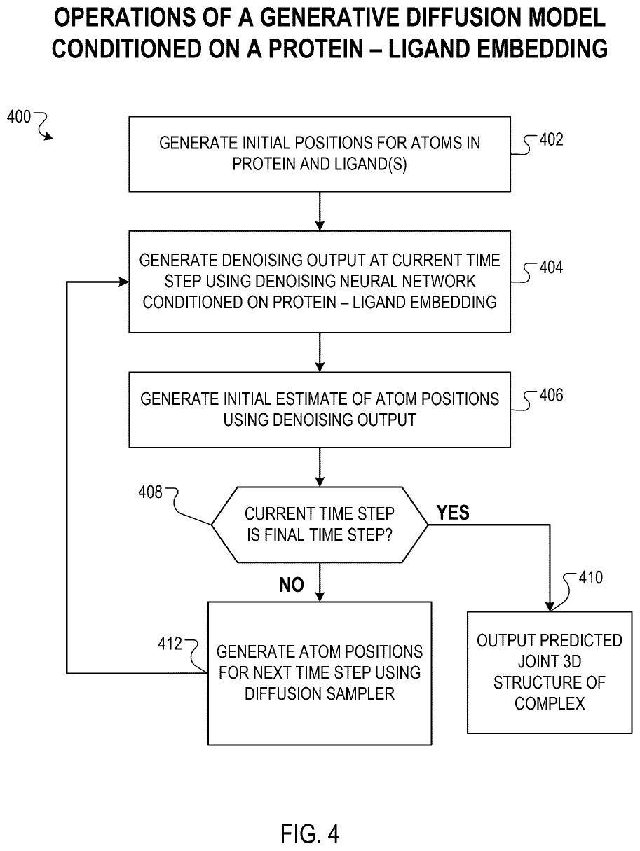

The generative model 108, when conditioned on the protein-ligand embedding 106, is configured to generate one or more predicted joint 3D structures 110 of the complex. The generative model 108 can be any appropriate conditional generative model. More specifically, the generative model 108 can any appropriate model that, when conditioned on the protein-ligand embedding 106, can generate samples from a distribution over a space of possible joint 3D structures of the complex. For instance, the generative model 108 can be implemented as a generative diffusion model, or a generative adversarial neural network (GAN) model, or a flow-based neural network model (normalizing flow model), and so forth. An example process for generating predicted joint 3D structures using a generative diffusion model is described detail with reference to FIG. 4-FIG. 6.

In some implementations, the generative model 108 can be implemented as an equivariant diffusion model, e.g., that is configured to perform operations that preserve symmetries such as rotational and translational invariance. An example of an equivariant diffusion model is described with reference to Hoogeboom, Emiel, et al. “Equivariant diffusion for molecule generation in 3d.” International conference on machine learning. PMLR, 2022.

Optionally, the generative model 108 can generate multiple distinct predicted joint 3D structures of the complex. In particular, the generative model 108 can generate multiple samples from the distribution over the space of possible joint 3D structures of the complex. Differences between the predicted joint 3D structures generated by the generative model 108 can reflect both uncertainty in the predicted structure and also the various structural modes (e.g., conformations) of a complex that includes the protein and the one or more ligands.

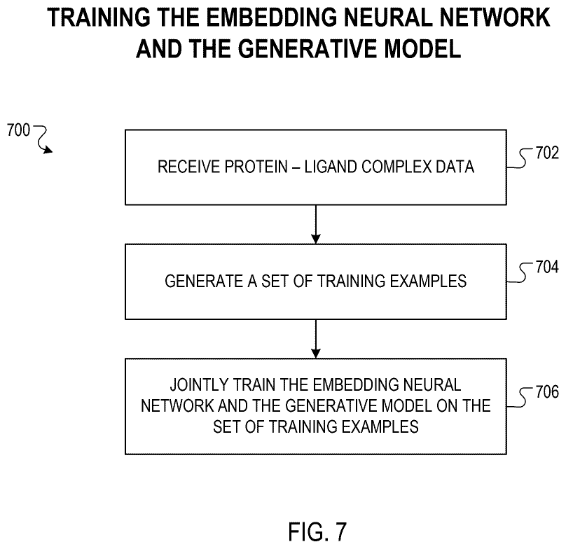

The cofolding system 100 can jointly train the embedding neural network 200 and the generative model 108 on a set of training data using an appropriate machine learning training technique. The training data can include a set of training examples, where each training example corresponds to a complex of a protein and one or more ligands, e.g., where the one or more ligands are each bound to a respective binding site on the protein. An example process for jointly training the embedding neural network 200 and the generative model 108 is described in more detail with reference to FIG. 7.

The cofolding system 100 can receive the protein data 102 and the ligand data 104 from any appropriate source, e.g., from a user or from another system, by way of an appropriate interface, e.g., an application programming interface (API) or a user interface (e.g., a graphical user interface). After generating the predicted joint 3D structure 110, the cofolding system 100 can, e.g., store data defining the predicted joint 3D structure 110 in a memory, or transmit data defining the predicted joint 3D structure 110 over a data communication network, or provide data defining the predicted joint 3D structure 110 directly to a system that performs downstream processing based on the predicted joint 3D structure 110.

Predicted joint 3D structures 110 generated by the cofolding system 100 can be used in any of a variety of possible downstream applications. A few examples of downstream applications that process predicted joint 3D structures 110 generated by the cofolding system 100 are described next.

In one example, the predicted joint 3D structure 110 (or features derived from the predicted joint 3D structure 110) can be processed by a scoring function to generate a predicted binding affinity of the one or more ligands for the protein. In some cases, the scoring function can generate the predicted binding affinity based on factors such as an electrostatic interaction factor, a van der Waals forces factor, a hydrophobic interaction factor, a lipophilic interaction factor, and so forth, that are each derived from the predicted joint 3D structure. In some cases, the scoring function can be a machine learning model that is configured to process the predicted joint 3D structure (or features derived from the predicted joint 3D structure) in accordance with values of a set of machine learning model parameters to generate the predicted binding affinity.

In some cases, predicted joint 3D structures 110 generated by the cofolding system 100 can be used for drug discovery. Drug discovery can involve identifying specific molecules within the body that are involved in a human or animal disease process. These molecules are often proteins, such as enzymes, receptors, or signaling proteins, that play a key role in the disease's development or progression. A ligand, often a small molecule (e.g., with a molecular weight equal to or less than 900 daltons), peptide, or antibody, can be selected to bind specifically to an identified target protein. When a drug that includes the ligand is administered to a patient, the ligand can bind to the target protein with high affinity and in doing so contribute to achieving a therapeutic effect in the patient. For instance, if the target protein is an enzyme involved in a disease process, the ligand can inhibit its activity, thus disrupting the disease pathway. More generally, the interaction between the ligand and the target protein can activate, inhibit, or alter the function of the target protein to achieve a therapeutic effect. For example, the ligand can be an agonist or antagonist of a receptor of the target protein.

Therefore, identifying (e.g., characterizing or defining) ligands with high (or low) binding affinity for a protein can be a crucial step in the process of drug discovery. (Identifying ligands with low binding affinities for a protein can be desirable, e.g., when the protein is an off-target protein and the binding of the ligand to the protein may cause undesirable side effects). However, determining binding affinities of ligands for proteins, e.g., through computational simulations or physical experiments, can be expensive and time consuming.

To address these issues, predicted joint 3D structures 110 generated by the cofolding system 100 can be used to determine a ranking of candidate ligands in a collection of candidate ligands based on their respective predicted binding affinities for a protein. More specifically, for each candidate ligand, the cofolding system 100 can generate a respective predicted joint 3D structure 110 of the protein and the candidate ligand. For each candidate ligand, a scoring function can process the predicted joint 3D structure 110 of the protein and the candidate ligand to generate a predicted binding affinity of the candidate ligand for the protein. The collection of candidate ligands can then be ranked based on the respective predicted binding affinity of each candidate ligand for the protein. The collection of candidate ligands can be obtained from an existing library of ligands, such as so-called compound libraries (e.g. available commercially), libraries generated by combinatorial techniques, and other sources (e.g. databases of complexes, such as the protein data bank).

The ranking of the candidate ligands in the collection of candidate ligands based on their respective predicted binding affinities for the protein can be used, e.g., to select a proper subset of the collection of candidate ligands for experimental validation and testing. For instance, one or more candidate ligands having the highest or lowest predicted binding affinities for the protein (i.e., according to the ranking) can be selected for experimental validation and testing, e.g., for use in a drug that achieves a therapeutic effect in patients. For example, each selected candidate ligand can be physically synthesized and then tested, e.g., in vitro (such as in a cell culture or tissue model) or in vivo, for a variety of properties, e.g., absorption, distribution, metabolism, and/or excretion, by a living organism or cell culture or tissue model. For example the selected ligand(s) can be screened according to a degree to which binding is accompanied by a biological (therapeutic) effect such as facilitating a biological mechanism or directly or indirectly inhibiting a biological disease mechanism (e.g. inhibiting a bacteria or virus from entering a cell), toxicity, clearance time, and so forth. One or more of the candidate ligands from the collection of ligands can be selected for inclusion in a drug, e.g., based at least in part on results of the testing. A drug that includes one or more of the candidate ligands can be synthesized using any appropriate drug synthesis technique.

The collection of candidate ligands can include any appropriate number of ligands, e.g., 10 ligands, or 1000 ligands, or 100,000 ligands. In some cases, only a fraction of the candidate ligands in the set of candidate ligands are selected for physical synthesis, e.g., based on the ranking of the candidate ligands by their predicted binding affinities for the protein. For instance, less than 50%, or less than 10%, or less than 1%, or less than 0.1% of the candidate ligands in the collection of candidate ligands may be selected for physical synthesis.

In some cases, predicted joint 3D structures 110 generated by the cofolding system 100 can be used for drug repurposing. In more detail, an existing drug may include a particular ligand, e.g., that is known to achieve a therapeutic effect in patients by binding to a target protein involved with a particular disease process. Drug repurposing can involve identifying new protein binding targets for the ligand, e.g., that are potentially involved in different disease processes. If the ligand has a high binding affinity for a new target protein that is involved in a disease process, then the ligand can be selected for experimental validation and potential inclusion in a drug for treating the disease. Drug repurposing can leverage the safety and efficacy data already available for a drug that includes the ligand, potentially accelerating the development process and reducing research costs. Drug repurposing can identify novel treatment options and address unmet medical needs by repurposing known ligands to treat different diseases or conditions.

To identify new target proteins for a ligand, predicted joint 3D structures 110 generated by the cofolding system 100 can be used to determine a ranking of candidate proteins in a collection of candidate proteins based on a respective predicted binding affinity of a particular ligand for each of the candidate proteins. More specifically, for each candidate protein, the cofolding system 100 can generate a respective predicted joint 3D structure 110 of the ligand and the candidate protein. For each candidate protein, a scoring function can process the predicted joint 3D structure 110 of the ligand and the candidate protein to generate a predicted binding affinity of the ligand for the candidate protein. The collection of candidate proteins can then be ranked based on the respective predicted binding affinity of the ligand for each of the candidate proteins.

The ranking of the candidate proteins in the collection of candidate proteins based on the predicted binding affinities of the ligand for the candidate proteins can be used, e.g., to select a proper subset of the collection of candidate proteins for experimental validation and testing. For instance, one or more candidate proteins for which the ligand has the highest predicted binding affinity (i.e., according to the ranking) can be selected for experimental validation and testing, e.g., for use in a drug that achieves a therapeutic effect in patients. In particular, each selected candidate protein can be physically synthesized and the binding affinity of the ligand for the candidate protein can then be experimentally tested and validated. One or more of the candidate proteins from the collection of candidate proteins can be selected as binding targets for the ligand, e.g., based at least in part on results of the testing.

The collection of candidate proteins can include any appropriate number of proteins, e.g., 10 proteins, or 1000 proteins, or 100,000 proteins. In some cases, only a fraction of the candidate proteins in the set of candidate proteins are selected for physical synthesis, e.g., based on the ranking of the candidate proteins by the predicted binding affinity of the ligand for the candidate proteins. For instance, less than 50%, or less than 10%, or less than 1%, or less than 0.1% of the candidate proteins in the collection of candidate proteins may be selected for physical synthesis.

The cofolding system and methods described herein can be used to obtain a ligand (i.e. a ligand molecule or ligand molecule complex) such as a drug or a ligand of an industrial enzyme. In general, the drug or industrial enzyme may be a molecule that inhibits or catalyzes a chemical or biochemical process. The molecule complex may include, for example, a protein, a ribozyme (ribonucleic acid enzyme), or a deoxyribozyme (deoxyribonucleic acid enzyme). For example, a method of obtaining a ligand may include obtaining a target amino acid sequence, in particular the amino acid sequence of a target protein molecule (or target protein molecule complex), e.g. a drug target, and processing an input based on the target amino acid sequence using the cofolding system to determine a (tertiary) structure of the target protein molecule, e.g., a predicted structure of a complex comprising the target protein molecule and the candidate ligand. The method may then include evaluating an interaction of one or more candidate ligands with the target protein molecule. The method may further include selecting one or more of the candidate ligands as the ligand dependent on a result of the evaluating of the interaction. Predicting the structure of a complex comprising the target protein molecule and a candidate ligand may preferably account for changes in the structure of molecule caused by binding of the candidate ligand and/or changes in the structure of the candidate ligand. Evaluating the interaction of the one or more candidate ligands with the target protein molecule may, for example, comprise determining a binding energy or an equilibrium constant for the formation of the complex.

In some implementations, evaluating the interaction may include evaluating binding of the candidate ligand with the structure of the target protein molecule. For example, evaluating the interaction may include identifying a ligand that binds with sufficient affinity for a biological effect. In some other implementations, evaluating the interaction may include evaluating an association of the candidate ligand with the target protein molecule which has an effect on a function of the target protein molecule, e.g., an enzyme. The evaluating may include evaluating an affinity between the candidate ligand and the target protein molecule or complex, or evaluating a selectivity of the interaction. The candidate ligand(s) may be selected according to which have the highest affinity. Evaluating the interaction may additionally comprise simulating a dynamical behavior of the ligand and target protein molecule, such as through molecular dynamics simulations, which may allow kinetic aspects of the interaction to be taken into account.

The candidate ligand(s) may be derived from a database of candidate ligands, and/or may be derived by modifying ligands in a database of candidate ligands, e.g., by modifying a structure or amino acid sequence of a candidate ligand, and/or may be derived by stepwise or iterative assembly/optimization of a candidate ligand. The candidate ligand(s) may alternately or additionally include one or more candidate ligands generated using a generative model conditioned on (the structure of) the target protein molecule or part of the target protein molecule, e.g. a structure of a binding site or other part of the target protein molecule.

The evaluation of the interaction of a candidate ligand with the target protein molecule may be performed using a computer-aided approach in which graphical models of the candidate ligand and target protein molecule structure are displayed for user-manipulation, and/or the evaluation may be performed partially or completely automatically, for example using standard molecular (e.g. protein-ligand) docking software. In some implementations the evaluation may include determining an interaction score for the candidate ligand, where the interaction score includes a measure of an interaction between the candidate ligand and the target protein molecule. The interaction score may be dependent upon a strength and/or specificity of the interaction, e.g., a score dependent on binding free energy. A candidate ligand may be selected dependent upon its score.

In some implementations the target protein molecule includes a receptor or enzyme and the ligand is an agonist or antagonist of the receptor or enzyme. In some implementations the method may be used to identify the structure of a cell surface marker. This may then be used to identify a ligand, e.g., an antibody or aptamer or a label such as a fluorescent label, which binds to the cell surface marker. This may be used to identify and/or treat cancerous cells.

In some implementations the ligand is a drug and the interaction of each of a plurality of target protein molecules with each of the candidate ligands is evaluated. Then one or more of the candidate ligands may be selected either to obtain a ligand that (functionally) interacts with each of the target protein molecules, or to obtain a ligand that (functionally) interacts with only one of the target protein molecules. For example in some implementations it may be desirable to obtain a drug that is effective against multiple drug targets. Also or instead it may be desirable to screen a drug for off-target effects. For example in agriculture it can be useful to determine that a drug designed for use with one plant species does not interact with another, different plant species and/or an animal species.

In some implementations the ligand is a drug and the predicted structure of a target protein that is a protein complex, e.g. a dimer or multimer, is determined. Evaluating the interaction of the one or more candidate ligands with the target protein may then comprise identifying a candidate ligand that interacts with the protein complex, and that might therefore be expected to affect the formation or stability of the complex. This could afterwards be confirmed by experimental screening. Thus such a process may be used to identify a drug which is able to disrupt a protein complex or inhibit formation of the complex. Some diseases, e.g. neurodegenerative diseases such as dementia, are caused by protein aggregation. The method may thus be used to identify a ligand that is a drug to treat such a disease.

In some implementations the candidate ligand(s) may include small molecule complex ligands, e.g., organic compounds with a molecular weight of <900 daltons. In some other implementations the candidate ligand(s) may include polypeptide ligands, i.e., defined by an amino acid sequence.

In another aspect there is provided a method of using the cofolding system to obtain a ligand, which may be a biological molecule, such as a polypeptide, polynucleotide, or polynucleoside ligand (e.g., the molecule or its amino acid or nucleotide sequence). For example, the method may include obtaining data defining one or more candidate ligands, e.g. an amino acid sequence of one or more candidate polypeptide or polynucleotide ligands. The method may include selecting a target molecule to which the ligand is to bind. The method may further include, for each of the candidate ligands, using the cofolding system to determine (tertiary) structure of a complex comprising the candidate ligand and the target protein molecule. The method may further include obtaining a target protein structure of a target molecule, in silico and/or by physical investigation. The method may comprise evaluating an interaction between each of the one or more candidate ligands and the target protein molecule, e.g. by evaluating an interaction between the predicted structure of the candidate ligand and the structure of the target molecule, or using the predicted structure of the complex comprising the candidate ligand and the target protein molecule. The method may further include selecting one or more of the candidate ligands as the ligand dependent on a result of the evaluation.

As before, evaluating the interaction may include evaluating binding of the candidate ligand with the structure of the target protein molecule, e.g., identifying a ligand that binds with sufficient affinity for a biological effect, and/or evaluating an association of the candidate ligand with the structure of the target protein molecule which has an effect on a function of the target protein molecule, e.g., an enzyme, and/or evaluating an affinity between the candidate ligand and the structure of the target protein molecule, or evaluating a selectivity of the interaction. In some implementations the ligand may be an aptamer. Again the candidate ligand(s) may be selected according to which have the highest affinity.

As before, the selected ligand (e.g. selected polypeptide or polynucleotide ligand) may comprise a receptor or enzyme and the ligand may be an agonist or antagonist of the receptor or enzyme. In some implementations the ligand may comprises an antibody or aptamer and the target protein molecule comprises an antibody or aptamer target, for example a virus, in particular a virus coat protein, or a protein expressed on a cancer cell. In these implementations the antibody or aptamer binds to the antibody or aptamer target to provide a therapeutic effect. For example, the antibody or aptamer may bind to the target and act as an agonist for a particular receptor; alternatively, the antibody or aptamer may prevent binding of another ligand to the target, and hence prevent activation of a relevant biological pathway.

Implementations of the method may further include synthesizing the ligand, i.e., making, the small molecule, polynucleotide or polypeptide ligand. The ligand may be synthesized by any conventional chemical techniques and/or may already be available, e.g., may be from a compound library or may have been synthesized using combinatorial chemistry.

The method may further include testing the ligand for biological activity in vitro and/or in vivo. For example the ligand may be tested for ADME (absorption, distribution, metabolism, excretion) and/or toxicological properties, to screen out unsuitable ligands. The testing may include, e.g., bringing the candidate small molecule, polypeptide or polynucleotide ligand into contact with the target protein molecule and measuring a change in expression or activity of the target molecule.

In some implementations a candidate (e.g. polypeptide or polynucleotide) ligand may include: an isolated antibody or aptamer, a fragment of an isolated antibody or aptamer, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may include an antibody with a mutated or chemically modified amino acid Fc region, e.g., which prevents or decreases ADCC (antibody-dependent cellular cytotoxicity) activity and/or increases half-life when compared with a wild type Fc region. Candidate (polypeptide or polynucleotide) ligands may include antibodies with different CDRs (Complementarity-Determining Regions).

As another example, the target protein molecule may be an enzyme comprising a CRISPR associated protein and the ligand may comprise a guide RNA molecule. The method may be performed to identify a combination of guide RNA molecule and CRISPR associated protein, in particular one that operates efficiently to edit genes. Such a method can involve determining a predicted structure of the enzyme, e.g. as described above, in particular to check that the enzyme shape and the guide RNA shape fit and work together effectively. The guide RNA may have a part with a defined 3D structure, e.g. it may be a single guide RNA (sgRNA), incorporating a guide sequence and a tracrRNA sequence.

The cofolding system described herein can also be used to obtain a diagnostic antibody or aptamer marker of a disease. There is also provided a method that comprises selecting a target protein molecule that is to be recognized by the antibody or aptamer marker, and for each of one or more candidate antibodies or aptamers e.g. as described above, uses the cofolding system to determine a predicted structure of a complex comprising the target protein molecule and the candidate antibody or aptamer. The method may also involve, evaluating an interaction between each of the one or more candidate antibodies or aptamers and the target protein molecule, and selecting one of the one or more of the candidate antibodies or aptamers as the diagnostic antibody or aptamer marker dependent on a result of the evaluating, e.g. selecting one or more candidate antibodies or aptamers that have the highest affinity to the target protein. The method may include making the diagnostic antibody or aptamer marker. The diagnostic antibody or aptamer marker may be used to diagnose a disease by detecting whether it binds to the target protein molecule in a sample obtained from a patient, e.g. a sample of bodily fluid. As described above, a corresponding technique can be used to obtain a therapeutic antibody or aptamer (e.g. polypeptide or polynucleotide ligand).

In some other aspects a computer-implemented method as described above or herein may be used to identify active/binding/blocking sites on a target protein from its amino acid sequence.

The folding systems and methods described herein can also be used to determine the structure of a molecule or molecule complex. For example, an experimental technique may be applied to a physical sample comprising a molecule complex (i.e. the physical sample of the molecule complex is “interrogated” using the experimental technique) to measure experiment signals dependent on a structure of the molecule complex. In some implementations, the experimental technique may be a scattering technique or a spectroscopic technique. The experimental technique may, for example, comprise one or more of: x-ray crystallography, nuclear magnetic resonance (NMR), and electron microscopy (e.g. cryogenic electron microscopy, cryo-EM). A cofolding system may be used to determine a predicted structure of the molecule complex. The experiment signals may then be compared with corresponding simulated signals generated using the predicted structure of the molecule complex. For example, the predicted structure of the molecule complex can be used to generate predicted x-ray diffraction patterns (e.g. from an electron density distribution determined using the predicted structure) that can be compared with experimentally measured x-ray diffraction patterns. For example, the experiment signals may comprise NMR signals, electron microscope images, or x-ray diffraction patterns; or signals derived therefrom.

The predicted structure of the molecule complex may be determined by adjusting the predicted structure of the molecule complex dependent upon a result of the comparison. The method may be performed iteratively, wherein for each of one or more iterations, after the predicted structure has been adjusted, the predicted signals may be generated for the adjusted structure, and the comparing and adjusting performed again to refine the predicted structure. Alternatively (or additionally), a plurality of different possible structures of the molecule complex can be predicted and the expected experiment signals from each compared with the actual experiment signals to determine a match, e.g. a best or most likely match, that can be taken as the determined structure of the molecule complex.

In general an aptamer as described above may comprise DNA or RNA. An enzyme as described above may comprise a protein or a DNA enzyme, e.g. a deoxyribozyme or “DNAzyme”, or an RNA enzyme, e.g. a ribozyme. As well as the applications described above such enzymes can also be used for biosensors of many types, e.g. DNAzymes and aptamers can be useful for detecting metal ions, and in general aptamer targets can include small molecule complexes, proteins, and cells. Aptamers have many uses including, e.g. as probes in assays, as biosensors (e.g. they can detectably change shape when binding to a target), to modulate the activity of biomolecule complexes, and to provide a controlled release mechanism. The techniques described herein can be used to design, and then make, such aptamers, sometimes referred to as chemical antibodies.

FIG. 2 shows an example embedding neural network 200, e.g., that is included in the cofolding system described with reference to FIG. 1. The embedding neural network 200 is configured to process: (i) protein data 102 characterizing a protein, and (ii) ligand data 104 characterizing one or more ligands, to generate a protein-ligand embedding 106 of the protein and the one or more ligands.

The embedding neural network 200 includes a protein embedding neural network 202, a ligand embedding neural network 204, and a fusion neural network 210, which are each described in more detail next (and throughout this specification).

The protein embedding neural network 202 is configured to process the protein data 102 characterizing the protein to generate a protein embedding 206 of the protein. The protein embedding 206 can include, e.g., a respective amino acid embedding of each amino acid in each amino acid sequence of the protein.

The protein embedding neural network 202 can have any appropriate neural network architecture that enables the protein embedding neural network 202 to perform its described functions. In particular, the protein embedding neural network 202 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

A particular example of a possible architecture of the protein embedding neural network 202 is the “Evoformer” neural network described in Jumper et al., “Highly accurate protein structure prediction with AlphaFold,” Nature, Vol 596, 26 Aug. 2021. The Evoformer can process a network input derived from: (i) the amino acid sequence of a protein, (ii) an MSA for the protein, and (iii) the 3D structures of one or more template amino acid sequences, to generate an output that includes a “single representation” that defines a respective embedding of each position in each amino acid sequence of the protein. (A template amino acid sequence is an amino acid sequence for an amino acid chain in the protein where the folded structure of the template sequence is known, e.g., from physical experiments).

The ligand embedding neural network 204 is configured to process the ligand data 104 characterizing the one or more ligands to generate a respective ligand embedding 208 of each of the one or more ligands. In particular, the ligand embedding neural network 204 can be configured to separately process ligand data 104 characterizing each ligand to generate a respective embedding of each ligand. The embedding of each ligand can include a respective atom embedding representing each atom in the ligand. The embeddings of the individual ligands can jointly define the overall ligand embedding 208. For instance, the overall ligand embedding can be defined by concatenating the embeddings of the individual ligands. Thus, the overall ligand embedding can include a respective atom embedding for each atom in each ligand of the one or more ligands.

The ligand embedding neural network 204 can have any appropriate neural network architecture that enables the ligand embedding neural network 204 to perform its described functions. In particular, the ligand embedding neural network 204 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

In a particular example, the ligand embedding neural network 204 can be configured to receive a collection of initial atom embeddings that includes a respective initial atom embedding for each atom in a ligand and that is derived from data characterizing the ligand, e.g., a SMILES string representing the ligand. The initial atom embedding of each atom can include data characterizing the type of the atom, the other atoms to which the atom is bonded, whether the atom is included in any functional groups, and so forth. The ligand embedding neural network can process the collection of initial atom embeddings by a sequence of one or more attention neural network layers, that are each configured to update the collection of current atom embeddings by a self-attention operation, to generate the embedding of the ligand, e.g., as the collection of atom embeddings output by a final attention layer in the sequence of attention layers.

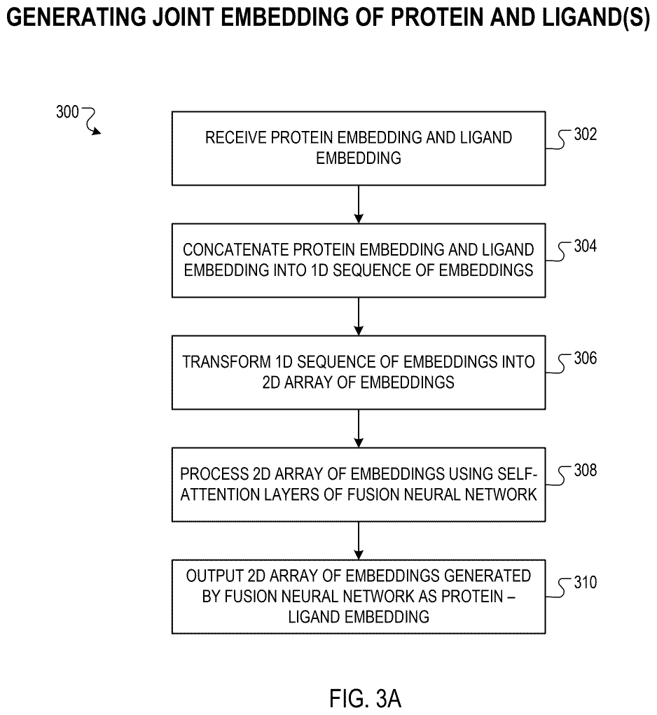

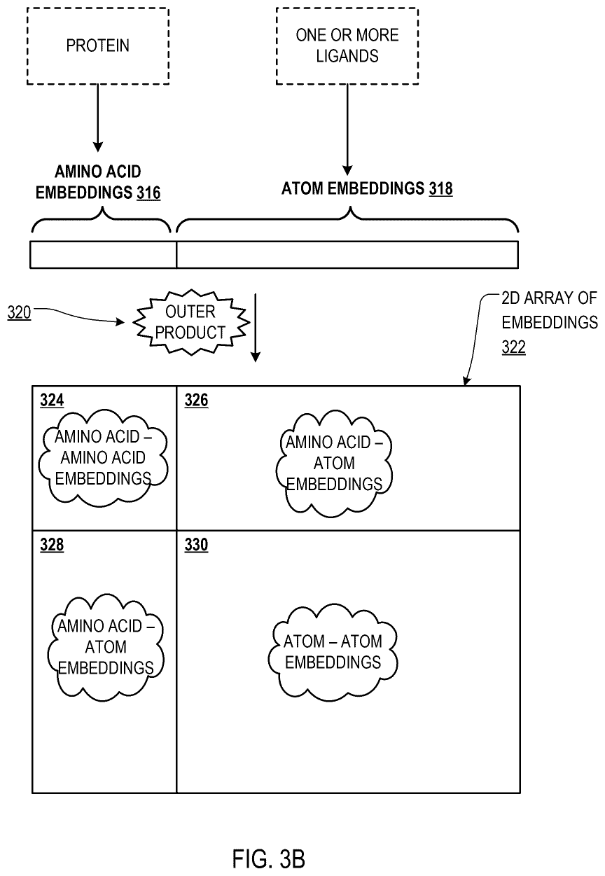

The fusion neural network 210 is configured to process the protein embedding 206 and the ligand embedding 208 to generate the protein-ligand embedding 106 that jointly represents the protein and the one or more ligands. The fusion neural network 210 can have any appropriate neural network architecture that enables the fusion neural network 210 to perform its described functions. In particular, the fusion neural network 210 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers). An example of processing a protein embedding 206 and a ligand embedding 208 to generate a protein-ligand embedding 106 is described in more detail with reference to FIG. 3A.

FIG. 3A is a flow diagram of an example process 300 for processing a protein embedding of a protein and a ligand embedding of one or more ligands using a fusion neural network to generate a protein-ligand embedding that jointly represents the protein and the one or more ligands. For convenience, the process 300 will be described as being performed by a system of one or more computers located in one or more locations. For example, a cofolding system, e.g., the cofolding system 100 of FIG. 1, appropriately programmed in accordance with this specification, can perform the process 300.

The system receives a protein embedding of a protein and a ligand embedding of one or more ligands (302). The protein embedding can be generated by a protein embedding neural network, as described with reference to FIG. 2, and can include a respective amino acid embedding for each position in each amino acid sequence of the protein. The ligand embedding can be generated by a ligand embedding neural network, as described with reference to FIG. 2, and can include a respective atom embedding for each atom in each ligand of the one or more ligands.

The system concatenates the protein embedding and the ligand embedding to generate a one-dimensional (1D) sequence of embeddings (304). The 1D sequence of embedding includes the amino acid embeddings of the protein embedding and the atom embeddings of the ligand embedding. The embeddings included in the 1D sequence of embeddings can be ordered in any appropriate way, e.g., the 1D sequence of embeddings can be ordered to have the amino acid embeddings followed by the atom embeddings, or to have the atom embeddings followed by the amino acid embeddings. The length of the 1D sequence of embeddings can be a sum of: (i) the number of amino acid embeddings in the protein embedding, and (ii) the number of atom embeddings in the ligand embedding. The 1D sequence of embeddings can be represented by data having dimensionality NumTokens×d, where NumTokens is given by the sum of: (i) the number of amino acid embeddings in the protein embedding, and (ii) the number of atom embeddings in the ligand embedding, and d is a positive integer value defining the number of channel dimensions in each amino acid embedding and atom embedding.

The system processes the 1D sequence of amino acid embeddings and atom embeddings to generate data defining a two-dimensional (2D) array of embeddings (306). The 2D array of embeddings can be represented by data having dimensionality NumTokens×NumTokens×d′ where (as above) NumTokens is given by the sum of: (i) the number of amino acid embeddings in the protein embedding, and (ii) the number of atom embeddings in the ligand embedding, and d′ is a positive integer value defining the number of channel dimensions in each embedding (d′ can be equal to d, i.e., the number of channel dimensions in each amino acid embedding and atom embedding). The system can generate the 2D array of embeddings from the 1D sequence of embeddings in any of a variety of ways. For instance, the system can generate the 2D array of embeddings as a result of an element-wise outer product of the 1D sequence of embeddings with itself. As another example, the system can generate the 2D array by an appropriate 2D concatenation operation, e.g., where the embedding at each position (i, j) in the 2D array of embeddings is generated by concatenating: (i) the embedding at position i, and (ii) the embedding at position j, in the 1D sequence of amino acid embeddings and atom embeddings (where indices i, j∈{1, . . . . N}, where N is the length of the 1D sequence of embeddings).

Each embedding in the 2D array of embeddings can be, e.g.: (i) an atom-atom embedding, or (ii) an amino acid-amino acid embedding, or (iii) an amino acid-atom embedding. Each atom-atom embedding is derived from a pair of atom embeddings representing atoms in the one or more ligands. Each amino acid-amino acid embedding is derived from a pair of amino acid embeddings representing amino acids in the protein. Each amino acid-atom embedding is derived from a pair of embeddings that includes an amino acid embedding representing an amino acid in the protein and an atom embedding representing an atom in a ligand.

The system processes the 2D array of embeddings by a set of neural network layers of the fusion neural network to generate an updated 2D array of embeddings defining the protein-ligand embedding (308). The updated 2D array of embeddings can have the same dimensionality as the original 2D array of embeddings, e.g., NumTokens×NumTokens×d′ (where NumTokens and d′ are defined as above). To generate the updated 2D array of embeddings, the fusion neural network can process the 2D array of embeddings using a sequence of one or more self-attention blocks. Each self-attention block can be configured to receive the current 2D array of embeddings as an input, to update the current 2D array of embeddings by one or more self-attention operations (e.g., single-head or multi-head query-key-value (QKV) self-attention operations), and to provide the updated 2D array of embeddings to a subsequent neural network layer (e.g., to another self-attention block, or to an output layer of the fusion neural network).

The self-attention blocks of the fusion neural network can implement any appropriate self-attention operations. A few examples of self-attention operations that can be implemented by self-attention blocks of the fusion neural network are described next.

In some implementations, one or more of the self-attention blocks of the fusion neural network implement “row-wise” or “column-wise” self-attention over the current 2D array of embeddings (i.e., that is provided as an input to the self-attention block). In a row-wise self-attention operation, a self-attention layer updates each given embedding in the 2D array of embeddings using a self-attention operation over only embeddings located in the same row as the given embedding in the 2D array of embeddings. In a column-wise self-attention operation, a self-attention block updates each given embedding in the 2D array of embeddings using a self-attention operation over only embeddings located in the same column as the given embedding in the 2D array of embeddings.

In some implementations, one or more of the self-attention blocks of the fusion neural network implement triangle self-attention operations. An example implementation of triangle self-attention operations are described in Jumper et al., “Highly accurate protein structure prediction with AlphaFold,” Nature, Vol 596, 26 Aug. 2021.

In some implementations, one or more of the self-attention blocks of the fusion neural network implement a full self-attention operation over the current 2D array of embeddings, e.g., by updating each embedding in the 2D array of embeddings using attention over the entire 2D array of embeddings.

The fusion neural network can include other neural network layers, i.e., in addition to the sequence of self-attention blocks, e.g., other neural network layers (such as fully connected layers or normalization layers) that are interleaved among the self-attention blocks. The fusion neural network can also include features such as skip connections, e.g., to implement residual blocks in the fusion neural network.

The system outputs the 2D array of embeddings generated by the fusion neural network as the protein-ligand embedding (310). The system can provide protein-ligand embedding generated by the fusion neural network, e.g., for conditioning the generative model, as will be described in more detail below with reference to FIG. 4-FIG. 6.

FIG. 3B illustrates operations performed by the cofolding system to generate the protein-ligand embedding. The system generates a protein embedding that includes a sequence of amino acid embeddings 316 (one for each amino acid in each amino acid sequence of the protein) and a ligand embedding that includes a sequence of atom embeddings 318 (one for each atom in each of the one or more ligands). The cofolding system concatenates the sequence of amino acid embeddings and the sequence of atom embeddings into a 1D sequence of embeddings, and then transforms the 1D sequence of embeddings (e.g., by an outer product operation) into a 2D array of embeddings. The 2D array of embeddings can include: (i) atom-atom embeddings 330, (ii) amino acid-amino acid embeddings 324, and (iii) amino acid-atom embeddings 326, 328. Each atom-atom embedding is derived from a pair of atom embeddings representing atoms in the one or more ligands. Each amino acid-amino acid embedding is derived from a pair of amino acid embeddings representing amino acids in the protein. Each amino acid-atom embedding is derived from a pair of embeddings that includes an amino acid embedding representing an amino acid in the protein and an atom embedding representing an atom in a ligand. The cofolding system can process the 2D array of embeddings 322 using a sequence of one or more self-attention blocks (e.g., that implement row-wise attention, or column-wise attention, or triangle self-attention, or full self-attention) to generate the protein-ligand embedding.

FIG. 4 is a flow diagram of an example process 400 for generating a predicted joint 3D structure of a protein and one or more ligands using a generative diffusion model that includes a denoising neural network. For convenience, the process 400 will be described as being performed by a system of one or more computers located in one or more locations. For example, a cofolding system, e.g., the cofolding system 100 of FIG. 1, appropriately programmed in accordance with this specification, can perform the process 400.

The system generates a respective initial 3D spatial position for each atom in the complex comprising the protein and the one or more ligands (402). For instance, for each atom in the complex, the system can sample a 3D spatial position of atom from a probability distribution over 3D space, e.g., a standard Normal distribution over 3D space.

The system performs steps 404-406, which are described next, over a sequence of iterations that may be referred to as “time steps”. The description of steps 404-406 (and the related steps 408 and 412) which follows will reference a “current” time step for convenience; the current time step can be any time step in the sequence of time steps. The system can perform the steps 404-406 over any appropriate number of time steps, e.g., 3 time steps, 10 time steps, or 100 time steps. The number of time steps can be a predetermined number of time steps.

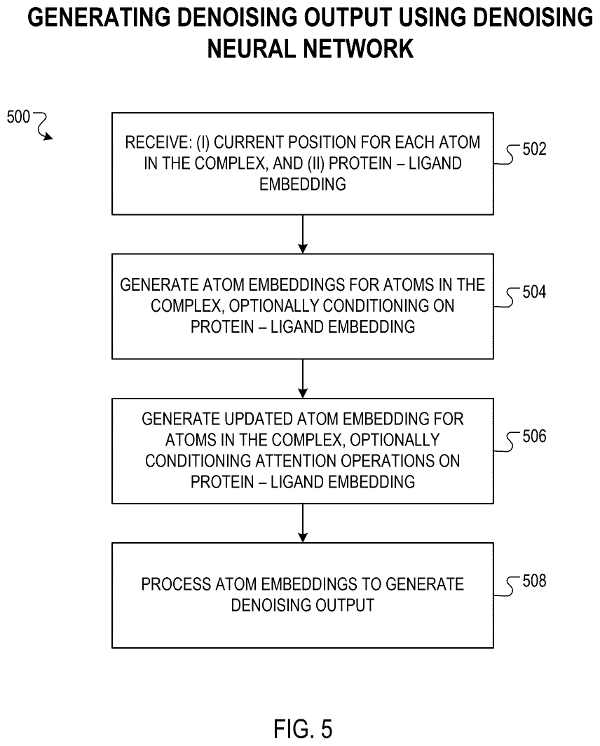

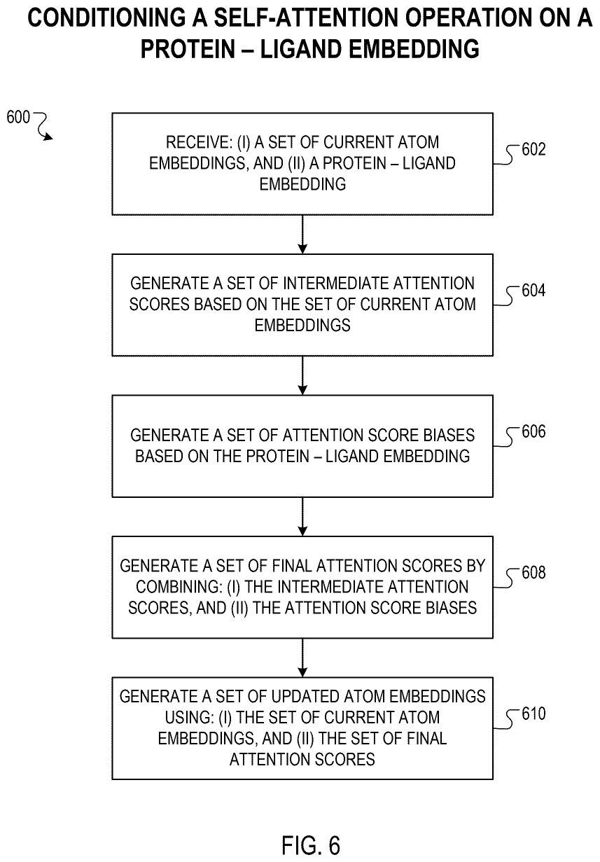

The system generates a denoising output using a denoising neural network that is conditioned on the protein-ligand embedding (404). The denoising output can be any appropriate data that enables estimation of the “final” 3D spatial position of each atom in the complex. For instance, the denoising output can define, for each atom in the complex, a predicted error in the 3D spatial position of the atom at the current time step. As another example, the denoising output can directly define, for each atom in the complex, a predicted 3D spatial position of the atom. As another example, the denoising output can define, for each atom in the complex, both: (i) a predicted error in the 3D spatial position of the atom at the current time step, and (ii) a predicted 3D spatial position of the atom. As another example, the denoising output can define, for each atom in the complex, a prediction for a value that is a linear combination of: (i) an actual 3D spatial position of the atom, and (ii) an error between the 3D spatial position of the atom at the current time step and the actual 3D spatial position of the atom, e.g., as implemented by the v-parametrization described in: Tim Salimans, Jonathan Ho, “Progressive distillation for fast sampling of diffusion models,” ICLR 2022, arXiv: 2202.00512v2. An example process for generating a denoising output using the denoising neural network is described in more detail with reference to FIG. 5-FIG. 6.

The system generates an initial estimate of the 3D spatial position for each atom in the complex using at least the denoising output generated by the denoising neural network (406). The system can generate the initial estimate of the 3D spatial position for each atom in the complex in any appropriate way, depending on the form of the denoising output. A few example techniques for generating the initial estimate of the 3D spatial position for each atom in the complex using the denoising output are described next.

In one example, the denoising output defines, for each atom, a respective prediction for the 3D spatial position of the atom. In this example, the respective predicted 3D spatial position for each atom defines the initial estimate of the 3D spatial position for the atom.

In another example, the denoising output defines, for each atom, a predicted error in the 3D spatial position of the atom at the current time step. In this example, the system can generate the initial estimate for the 3D spatial position for each atom as a linear combination of: (i) the current 3D spatial position of the atom, and (ii) the predicted error in the 3D spatial position of the atom. Each term in the linear combination can be scaled by a respective constant value that is dependent on the time step. For instance, the system can generate the initial estimate for the 3D spatial position xₜ₋₁ for an atom in the complex as:

$${x}_{t - 1} = {α}_{t}^{- 0.5} ( {x}_{t} - ( 1 - {α}_{t} ) {( 1 - {\overline{α}}_{t} )}^{- 0.5} {ϵ}_{θ} ( {x}_{t} , t ) ) \qquad ( 1 )$$

where t indexes the current time step, αₜ, α̅ₜ, and σₜ are constants specific to time step t, and ∈_{θ}(xₜ, t) is the predicted error in the 3D spatial position of the atom (e.g., as generated by the denoising neural network at the time step). (In the notation of equation (1), the time steps decrement, such that time step t-1 is the “next” time step after time step t). The constants in equation (1) (αₜ, α̅ₜ, and σₜ) can be selected in accordance with a predefined noise schedule.

In another example, the denoising output defines, for each atom, both: (i) a predicted 3D spatial position of the atom, and (ii) a predicted error in the 3D spatial position of the atom at the current time step. In this example, the system can generate the initial estimate for the 3D spatial position of the atom as a combination (e.g., an average) of: (i) the predicted 3D spatial position of the atom as specified by the denoising output, and (ii) a predicted 3D spatial position of the atom that is derived from the predicted error in the 3D spatial position of the atom at the current time step, e.g., using equation (1).

In another example, the denoising output is expressed using a v-parametrization, and the system generates a respective initial estimate for the 3D spatial position for each atom using the techniques described in Tim Salimans, Jonathan Ho, “Progressive distillation for fast sampling of diffusion models,” ICLR 2022, arXiv: 2202.00512v2.

Optionally, the system can generate a respective confidence measure for the initial estimate of the respective 3D spatial position of each atom in the complex. For instance, as part of generating the denoising output, the denoising neural network can generate a respective atom embedding for each atom in the complex, e.g., as the output of the update block of the denoising neural network, as described with reference to step 506 of FIG. 5. The system can process each atom embedding using one or more neural network layers (e.g., a combination of one or more of: fully connected layers, or attention layers, or pooling layers) to generate a respective confidence estimate for the initial estimate of the 3D spatial position of the atom(s) represented by the atom embedding. The confidence measure for the initial estimate of the 3D spatial position of an atom can characterize a predicted error in the initial estimate of the 3D spatial position of the atom.

Optionally, the system can generate a respective confidence measure for the initial estimates of the 3D spatial positions of pairs of atoms in the complex. For instance, for a first atom in the complex and a second atom in the complex, the system can process an atom embedding representing the first atom and an atom embedding representing the second atom (e.g., as generated by the update block of the denoising neural network) using one or more neural network layers (e.g., a combination of one or more of: fully connected layers, or attention layers, or pooling layers) to generate a confidence estimate for the initial estimates of the 3D spatial positions of the first atom and the second atom. The confidence measure can characterize, e.g., a predicted error in the relative 3D displacement of the first atom and the second atom.

Optionally, the system can generate a confidence measure for the structure of the protein as defined by the initial estimates of the 3D spatial positions of the atoms in the protein, e.g., by combining (e.g., summing or averaging) the confidence measures for the individual atoms included in the protein.

Optionally, for each ligand, the system can generate a confidence measure for the structure of the ligand as defined by the initial estimates of the 3D spatial positions of the atoms in the ligand, e.g., by combining (e.g., summing or averaging) the confidence measures for the individual atoms included in the ligand.

Optionally, for each ligand, the system can generate a confidence measure for the structure of an interface between the protein and the ligand, e.g., by combining (e.g., summing or averaging) the confidence measures of pairs of atoms included in the interface. A pair of atoms can be referred to as being included in the interface, e.g., if the pair includes: (i) a first atom included in the ligand, and (ii) a second atom included in the protein, where the relative displacement between the 3D spatial positions of the atoms is less than a threshold, e.g., 2 Angstroms, or 3 Angstroms, or 8 Angstroms. An example of generating a confidence measure for a pair of atoms is described below.

If the current time step is the final time step (i.e., in the sequence of denoising time steps), the system outputs the initial estimate of the 3D spatial position of each atom in the complex as the predicted joint 3D structure of the complex (410).

If the current time step is not the final time step, the system generates a respective 3D spatial position for each atom for the next time step based on the initial estimates of the 3D spatial positions of the atoms (as generated at step 406) using an appropriate diffusion sampling technique (412). A few examples of possible diffusion sampling techniques are described next.

In one example, the system can generate the 3D spatial position of each atom in the complex at the next time step by combining random noise with the initial estimate of the 3D spatial position of the atom. For instance, for each atom, the system can add respective random noise to the initial estimate of the 3D spatial position of the atom. The random noise can be sampled from a probability distribution over 3D space. The probability distribution over 3D space can vary based on the time step, e.g., such that the variance of the noise combined with the updated 3D spatial positions of the atoms decreases over the sequence of time steps.

As another example, the system generates the 3D spatial position of each atom in the complex at the next time step using a deterministic diffusion sampling technique, i.e., that does not rely on random noise. An example of a deterministic diffusion sampling technique is the denoising diffusion implicit model (DDIM), e.g., as described in: Jiaming Song, Chenlin Meng, Stefano Ermon, “Denoising diffusion implicit models,” ICLR 2021, arXiv: 2010.02502v4.

Optionally, the system can perform the process 400 multiple times to generate multiple predictions for the joint 3D structure of the complex. Each execution of the steps of the process 400 can result in the generation of a different predicted joint 3D structure of the complex, e.g., as a result of stochasticity in the random sampling performed to generate the initial positions of the atoms (at step 402), and in some cases, as a result of stochasticity in diffusion sampler (at step 412).

By directly predicting atom coordinates, i.e., determining the 3D spatial position of each atom in the complex, the system can reduce or eliminate the need for further processing of the 3D structure of the complex, e.g., to enforce stereochemical constraints. For example, the system can generate chemically plausible and sharply defined local structure, such as for the configurations of sidechains of the amino acids, without needing carefully tuned stereochemical losses or special handling of bonding patterns or computationally expensive molecular dynamics simulations.

In some implementations, the system performs the operations of the process 400 multiple times to generate a collection of multiple (distinct) predicted 3D structures of the complex. The system can generate any appropriate number of predicted 3D structures of the complex, e.g., at least 5, or at least 10, or at least 1000, or at least 10,000 predicted 3D structures of the complex. Optionally, the system can rank the predicted 3D structures based on a respective confidence measure for each of the predicted 3D structures. Example techniques for determining a confidence measure for a predicted 3D structure are described above. Optionally, the system may discard any predicted 3D structures having a confidence measure that is below a confidence measure threshold.

FIG. 5 is a flow diagram of an example process 500 for generating a denoising output using a denoising neural network conditioned on a protein-ligand embedding. For convenience, the process 500 will be described as being performed by a system of one or more computers located in one or more locations. For example, a cofolding system, e.g., the cofolding system 100 of FIG. 1, appropriately programmed in accordance with this specification, can perform the process 500.

The system receives: (i) data defining a respective current 3D spatial position of each atom in the complex, and (ii) the protein-ligand embedding (502). The protein-ligand embedding can include: (i) atom-atom embeddings, (ii) amino acid-amino acid embeddings, and (iii) amino acid-atom embeddings, as described in more detail above with reference to FIG. 3. The system can optionally receive additional inputs, e.g., an input defining a current time step in a diffusion process being implemented by a generative diffusion model, as described above with reference to FIG. 4.

The system generates a respective atom embedding for each atom in the complex, using an encoder block of the generative neural network, based at least in part on the current 3D spatial position of the atom (504). Optionally, the system can generate the atom embeddings for the atoms based at least in part on the protein-ligand embedding (and, optionally, the current time step in the diffusion process), e.g., in addition to the current 3D spatial positions of the atoms. For instance, for each atom, the system can generate the atom embedding of the atom based on both: (i) the current 3D spatial position of the atom, and (ii) a respective conditioning embedding selected from the collection of embeddings included in the protein-ligand embedding. For an atom included in an amino acid in the complex, the conditioning embedding for the atom can be the amino acid-amino acid embedding (i.e., from the protein-ligand embedding) of the amino acid that includes the atom (i.e., the amino acid-amino acid embedding corresponding to a pair of amino acids that includes two copies of the amino acid). For an atom included in a ligand in the complex, the conditioning embedding for the atom can be the atom-atom embedding (i.e., from the protein-ligand embedding) of the atom (i.e., the atom-atom embedding corresponding to a pair of atoms that includes two copies of the atom).

The system can generate the atom embedding for an atom based on the current 3D spatial position of the atom (and, optionally, a conditioning embedding for the atom) in any of a variety of possible ways. For instance, the system can generate the atom embedding for an atom by processing the 3D spatial position of the atom using an encoder block of the denoising neural network. As another example, the system can generate the atom embedding for an atom by processing both: (i) the 3D spatial position of the atom, and (ii) the conditioning embedding for the atom, using an encoder subnetwork of the denoising neural network. As another example, the system can generate the atom embedding for an atom by concatenating: (i) an output generated by an encoder subnetwork of the denoising neural network by processing the 3D spatial position of the atom, and (ii) the conditioning embedding for the atom.

The encoder block of the denoising neural network can include any appropriate types of neural network layers (e.g., fully connected layers, convolutional layers, and so forth) in any appropriate number (e.g., 1 layer, 3, layers or 5 layers) and connected in any appropriate configuration (e.g., as a directed graph of layers). In a particular example, the encoder block includes a sequence of fully connected neural network layers and is configured to, for each atom, process data defining the 3D spatial position of the atom and the conditioning embedding for the atom to generate the atom embedding of the atom.

In some implementations, for each amino acid in the protein, the system can generate one atom embedding that jointly represents all the atoms in the amino acid. Thus, in these implementations, the number of atom embeddings may be equal to a sum of: (i) the number of atoms in the one or more ligands, and (ii) the number of amino acids in the protein. The system can generate an atom embedding that jointly represents all the atoms in an amino acid in any appropriate way. For instance, the system can generate a respective atom embedding for each atom in the amino acid (as described above), and then combine the atom embeddings for the atoms in the amino acid, e.g., using a pooling operation (e.g., a max pooling or a summation pooling operation), or by processing the atom embeddings for the atoms in the amino acid using one or more neural network layers (e.g., fully connected layers or self-attention layers) to generate the embedding that jointly represents all the atoms in the amino acid. Generating atom embeddings that jointly represent all the atoms in an amino acid can significantly reduce the overall number of atom embeddings and thus reduce consumption of computational resources, e.g., memory and computing power, resulting from operations performed by an update block of the denoising neural network, as will be described next.

The system processes the atom embeddings for the atoms in the complex, using an update block of the denoising neural network, to generate a respective updated atom embedding for each atom in the complex (506). The update block of the denoising neural network layer can include a sequence of self-attention blocks. Each self-attention block can be configured to receive a respective current atom embedding for each atom in the complex, to apply a self-attention operation to the current atom embeddings of the atoms in the complex, and to provide the updated atom embeddings, e.g., for processing by a subsequent neural network layer.

Each self-attention block included in the update block can apply any appropriate self-attention operation to the current atom embeddings of the atoms in the complex, e.g., a single-head or multi-head query-key-value (QKV) self-attention operation. Optionally, the system can condition the self-attention operations of one or more of the self-attention blocks on the protein-ligand embedding. An example process for implementing a self-attention operation conditioned on the protein-ligand embedding is described in more detail with reference to FIG. 6.

The update block of the denoising neural network can include any appropriate number of self-attention blocks (e.g., 1 self-attention block, or 10 self-attention blocks, or 50 self-attention blocks) and can optionally include additional neural network layers of any appropriate type (e.g., fully connected layers, convolutional layers, and so forth) in any appropriate number (e.g., 1 layer, 3, layers or 5 layers) and connected in any appropriate configuration (e.g., interleaved with the self-attention blocks).

The system processes the updated atom embeddings (i.e., as generated by the update block of the denoising neural network) using a decoder block of the denoising neural network to generate the denoising output (508). Examples of denoising outputs are described above with reference to FIG. 4. The decoder block of the denoising neural network can include any appropriate types of neural network layers (e.g., fully connected layers, convolutional layers, and so forth) in any appropriate number (e.g., 1 layer, 3, layers or 5 layers) and connected in any appropriate configuration (e.g., as a directed graph of layers).

In a particular example, the decoder block can include a sequence of fully connected neural network layers that are configured to operate separately on each updated atom embedding to generate a predicted error in the 3D spatial position of the corresponding atom at the current time step, or to generate a predicted 3D spatial position of the corresponding atom, or both.

In implementations where one atom embedding jointly represents all the atoms in an amino acid (as described above with reference to step 504), the decoder block can process that (updated) atom embedding to generate respective denoising outputs for all the atoms in the amino acid. For instance, the decoder block can process an updated atom embedding that jointly represents all the atoms in an amino acid to generate a respective predicted error in the 3D spatial position of each atom in the amino acid, or to generate a respective 3D spatial position of each atom in the amino acid, or both.

FIG. 6 is a flow diagram of an example process 600 for updating a set of current atom embeddings using a self-attention operation that is implemented by a self-attention block of the denoising neural network and that is conditioned on a protein-ligand embedding. For convenience, the process 600 will be described as being performed by a system of one or more computers located in one or more locations. For example, a cofolding system, e.g., the cofolding system 100 of FIG. 1, appropriately programmed in accordance with this specification, can perform the process 600.

The system receives: (i) a set of current atom embeddings, and (ii) a protein-ligand embedding (602). The set of current atom embeddings includes a respective atom embedding for each atom in the complex. (In some cases, for each amino acid in the protein, all the atoms in the amino acid are jointly represented by one (respective) atom embedding, as described above with reference to FIG. 5). The set of current atom embeddings can be generated, e.g., by the encoder block of the denoising neural network or by a previous self-attention layer in the update block of the denoising neural network, as described above with reference to FIG. 5. The protein-ligand embedding can be generated by an embedding neural network, e.g., as described above with reference to FIG. 2-FIG. 3A. The protein-ligand embedding can include: (i) atom-atom embeddings, (ii) amino acid-amino acid embeddings, and (iii) amino acid-atom embeddings.

The system generates a set of intermediate attention scores based on the set of current atom embeddings (604). The set of intermediate attention scores includes a respective attention score for each pair of current atom embeddings from the set of current atom embeddings.

The system can generate the intermediate attention scores in any of a variety of possible ways. For instance, the system can generate a respective query embedding for each current atom embedding by processing the current atom embedding using a query neural network, e.g., as:

$$Q = {W}^{Q} \cdot E \qquad ( 3 )$$

where Q is a matrix where each column (or row) defines a respective query embedding, W^{Q} is a matrix of parameter values (defining the query neural network in this example), and E is a matrix where each column (or row) defines a respective current atom embedding. Further, the system can generate a respective key embedding for each current atom embedding by processing the current atom embedding using a key neural network, e.g., as:

$$K = {W}^{K} \cdot E \qquad ( 4 )$$

where K is a matrix where each column (or row) defines a respective key embedding, W^{K} is a matrix of parameter values (defining the key neural network in this example), and E is a matrix where each column (or row) defines a respective current atom embedding. The system can generate the intermediate attention scores based on the query embeddings and the key embeddings, e.g., as:

$$A = Q \cdot {K}^{T} \qquad ( 5 )$$

where A is a matrix of intermediate attention scores, Q is the matrix of query embeddings, and K is the matrix of key embeddings.

The system generates a set of attention score biases based on the protein-ligand embedding (606). The set of attention score biases includes a respective attention score bias for each pair of current atom embeddings from the set of current atom embeddings.

The system can generate the set of attention score biases in any of a variety of possible ways. For instance, for each pair of current atom embeddings, the system can generate the attention score bias for the pair of current atoms embeddings by processing a corresponding conditioning embedding selected from the collection of embeddings included in the protein-ligand embedding using a projection neural network.

For a pair of current atom embeddings that includes: (i) a first current atom embedding representing a first atom included in a ligand, and (ii) a second current atom embedding representing a second atom included in a ligand, the conditioning embedding can be the atom-atom embedding for the first atom and the second atom in the protein-ligand embedding.

For a pair of current atom embeddings that includes: (i) a first current atom embedding representing an atom included in an amino acid, and (ii) a second current atom embedding representing an atom included in a ligand, the conditioning embedding can be the amino acid-atom embedding corresponding to: (i) the amino acid that includes the first atom, and (ii) the second atom, in the protein-ligand embedding.

For a pair of current atom embeddings that includes: (i) a first current atom embedding representing an atom included in a first amino acid, and (ii) a second current atom embedding representing an atom included in a second amino acid, the conditioning embedding can be the amino acid-amino acid embedding corresponding to: (i) the first amino acid, and (ii) the second amino acid, in the protein-ligand embedding.

For a pair of current atom embeddings that includes: (i) a first current atom embedding that jointly represents the atoms in a first amino acid, and (ii) a second current atom embedding that jointly represents the atoms in a second amino acid, the conditioning embedding can be the amino acid-amino acid embedding corresponding to: (i) the first amino acid, and (ii) the second amino acid, in the protein-ligand embedding.

For a pair of current atom embeddings that includes: (i) a first current atom embedding that jointly represents the atoms in an amino acid, and (ii) a second current atom embedding that represents an atom included in a ligand, the conditioning embedding can be the amino acid-atom embedding corresponding to: (i) the amino acid, and (ii) the atom, in the protein-ligand embedding.

The projection neural network can have any appropriate neural network architecture that enables the projection neural network to perform its described functions, e.g., processing a conditioning vector to generate an attention score bias. In particular, the projection neural network can include any appropriate number of neural network layers (e.g., 1 layer, or 5 layers, or 10 layers) in any appropriate number (e.g., 1 layer, 3, layers or 5 layers) and connected in any appropriate configuration (e.g., as a directed graph of layers).

The system generates a set of final attention scores by combining: (i) the intermediate attention scores, and (ii) the attention score biases (608). The set of final attention scores includes a respective final attention score for each pair of current atom embeddings in the set of current atom embeddings. The system can generate the final attention score for a pair of current atom embeddings by combining (e.g., summing): (i) the intermediate attention score for the pair of current atom embeddings, and (ii) the attention score bias for the pair of current atom embeddings. Combining the intermediate attention scores, and the attention score biases can allow the protein-ligand embedding to control (i.e., modulate) information flow in the current atom embeddings. Optionally, the system can apply further processing operations to the set of final attention scores, e.g., by applying a soft-max operation to some or all of the final attention scores.

The system generates a set of updated atom embeddings using: (i) the set of current atom embeddings, and (ii) the set of final attention scores (610). For instance, to generate the set of updated atom embeddings, the system can generate a respective value embedding for each current atom embedding in the set of current atom embeddings by processing the current atom embedding using a value neural network, e.g., as:

$$V = {W}^{V} \cdot E \qquad ( 6 )$$

where V is a matrix where each column (or row) defines a respective value embedding, W^{V} is a matrix of parameter values (defining the value neural network in this example), and E is a matrix where each column (or row) defines a respective current atom embedding. The system can then generate the set of updated atom embeddings, e.g., as:

$${E}^{′} = V \cdot A \qquad ( 7 )$$

where each column (or row) of E′ defines a respective updated atom embedding, each column (or row) of V defines a respective value embedding, and A denotes the set of final attention scores arranged into a matrix.

In implementations where the self-attention block implements a multi-head attention operation, each head of the attention operation can individually perform the steps of the process 600, and the updated atom embeddings generated by each attention head can be combined (e.g., concatenated) to define the overall output of the multi-head attention operation. Each attention head can have a respective set of neural network parameters, having values that are specific to each attention head, that are used for generating the intermediate attention scores and the attention score biases.

FIG. 7 is a flow diagram of an example process 700 for jointly training the embedding neural network and the generative model of the cofolding system. In the example process 700, the generative model is a model that implements a differentiable generative process. For instance, the generative model can be a generative diffusion model implemented using a denoising neural network, as described above with reference to FIG. 4-FIG. 6. For convenience, the process 700 will be described as being performed by a system of one or more computers located in one or more locations. For example, a cofolding system, e.g., the cofolding system 100 of FIG. 1, appropriately programmed in accordance with this specification, can perform the process 700.

The system receives data characterizing a set of protein-ligand complexes (702). Each protein-ligand complex defines a joint 3D structure of a protein and one or more ligands, e.g., where each of the one or more ligands is bound to a respective binding site on the protein. The joint 3D structures of the received data may have been determined, e.g., through physical experiments. There are also many public databases that can be used, such as the Protein Data Bank (wwpdb.org), and many others. In some cases, joint 3D structures for training the system may also be obtained using other protein structure determination systems, e.g., the system described Jumper et al., “Highly accurate protein structure prediction with AlphaFold,” Nature, Vol 596, 26 Aug. 2021.

The system generates a set of training examples (704). Each training example corresponds to a respective protein-ligand complex and includes data defining: (i) a training input to the cofolding system, and (ii) a target output of the cofolding system. The training input to the cofolding system includes protein data (characterizing the protein of the protein-ligand complex) and ligand data (characterizing the one or more ligands of the protein-ligand complex). The target output of the cofolding system can be based on the joint 3D structure of the protein-ligand complex.

The system jointly trains the embedding neural network and the generative model on the set of training examples by a machine learning training technique (706). More specifically, for each training example, the system can process the training input of the training example using the embedding neural network and the generative model to generate a predicted output of the generative model. The system can evaluate an objective function that measures an error (e.g., a root mean square deviation (RMSD), or a mean absolute error (MAE), or a mean squared error (MSE)) between: (i) the predicted output of the generative model, and (ii) the target output of the generative model. The system can determine gradients of the objective function with respect to the parameters of the embedding neural network and the generative model, e.g., using backpropagation. (The parameters of the generative model can include, e.g., a set of neural network parameters of a neural network implemented by the generative model). The system can then update the current values of the parameters of the embedding neural network and the generative model using the gradients, e.g., by the update rule of an appropriate gradient descent optimization algorithm, e.g., RMSprop or Adam.

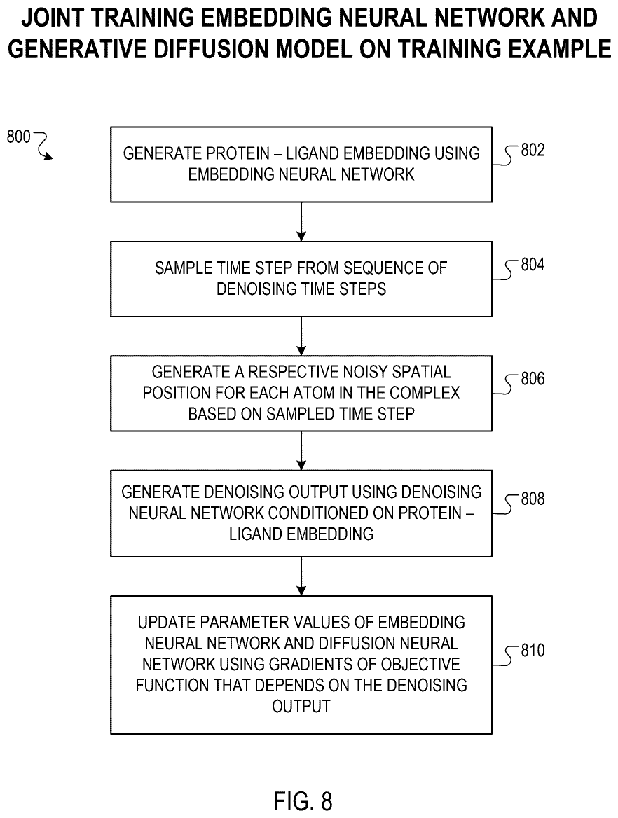

Specific aspects of the training (e.g., the operations of the generative model during the training and the objective function) may depend on the implementation of the generative model. An example process for training a generative diffusion model that includes a denoising neural network on a training example is described in more detail next with reference to FIG. 8. In the example process of FIG. 8, the operations of the generative diffusion model are modified during training (e.g., as compared to the operations of the generative diffusion model during inference, e.g., as described with reference to FIG. 4), as will be described in more detail below.

In some implementations, the generative model includes one or more “confidence estimation” neural network layers that process atom embeddings generated by the generative model to generate confidence measures for the estimated positions of atoms or pairs of atoms in a complex (as described above with reference to FIG. 4). The system can jointly train the confidence estimation neural network layers along with the embedding neural network and the generative model. For instance, for each atom in the complex, the system can generate a confidence measure for the atom that defines a predicted error in the 3D spatial position of the atom, and the objective function can include a term that measures a difference between: (i) the predicted error in the 3D spatial position of the atom, and (ii) the actual error in the 3D spatial position of the atom. As another example, for each pair of atoms in the complex, the system can generate a confidence measure that defines a predicted error in the relative 3D displacement of the pair of atoms, and the objective function can include a term that measures a difference between: (i) the predicted relative 3D displacement of the pair of atoms, and (ii) the actual relative 3D displacement of the pair of atoms. The system can backpropagate gradients through the confidence estimation neural network layers, and optionally, into the generative model and/or the embedding neural network.

FIG. 8 is a flow diagram of an example process 800 for jointly training an embedding neural network and a generative diffusion model on a training example. For convenience, the process 800 will be described as being performed by a system of one or more computers located in one or more locations. For example, a cofolding system, e.g., the cofolding system 100 of FIG. 1, appropriately programmed in accordance with this specification, can perform the process 800.

The system generates a protein-ligand embedding of the protein-ligand complex corresponding to the training example using the embedding neural network (802).

The system samples a time step from the sequence of denoising time steps (804). More specifically, during inference, the generative diffusion model can be configured to perform a sequence of denoising time steps, e.g., as described with reference to steps 404, 406, 408, and 412 of FIG. 4. During training, the system can randomly sample a single denoising time step from the sequence of denoising time steps, e.g., in accordance with a uniform distribution over the sequence of denoising time steps.

The system generates a respective noisy spatial position for each atom in the complex by combining random noise with the target spatial position of the atom (i.e., in the target 3D structure of the complex) (806). For instance, for each atom in the complex, the system can generate the noisy spatial position for the atom by adding random noise to the target spatial position of the atom. The system can scale the random noise combined with the target spatial positions of the atoms by a constant that depends on the sampled time step, e.g., where the values of the constants corresponding to the denoising time steps are defined by a noise schedule.

The system generates a denoising output using the denoising neural network while the denoising neural network is conditioned on the protein-ligand embedding (808). An example process for generating a denoising output is described in detail with reference to FIG. 5. At step 502 of FIG. 5, the current position for each atom in the complex can be defined as the noisy spatial position for each atom in the complex.

The system determines gradients of an objective function that depends on the denoising output, and uses the gradients to update the parameter values of the denoising neural network and the embedding neural network (810). The objective function can measure an error between: (i) the denoising output of the denoising neural network, and (ii) a target output of the denoising neural network. The target output of the denoising neural network can define an output of the denoising neural network that, if used to generate an initial estimate of the 3D spatial positions of the atoms in the complex (as described in step 406 of FIG. 4), would cause the initial estimate of the 3D spatial positions of the atoms to match the target (actual) 3D spatial positions of the atoms in the protein-ligand complex of the training example.

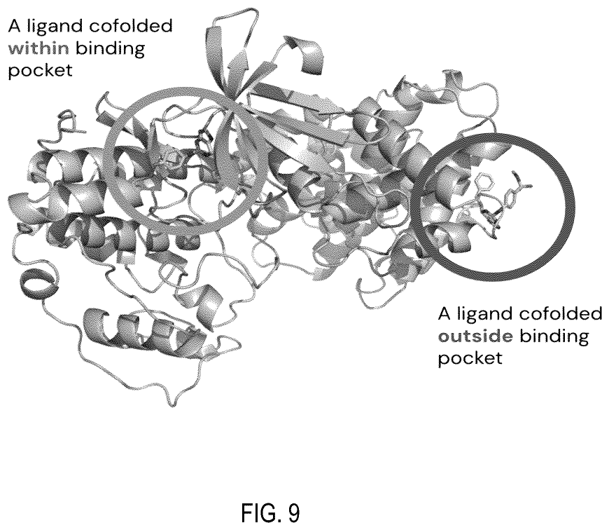

FIG. 9 illustrates an example of an output generated by the cofolding system showing a predicted 3D structure of a protein and two ligands. In this example, a first ligand is bound to a binding pocket of the protein, while a second ligand is outside any binding pockets of the ligand, e.g., indicating that the second ligand may not bind to any binding pockets on the protein, or that the second ligand has a lower binding affinity for the protein than the first ligand.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
obtaining a network input that characterizes a protein and one or more ligands;
processing the network input characterizing the protein and the one or more ligands using an embedding neural network to generate a protein-ligand embedding of the protein and the one or more ligands; and
generating, using a generative model and while the generative model is conditioned on the protein-ligand embedding, a predicted joint three-dimensional (3D) structure of the protein and the one or more ligands,
wherein the predicted joint 3D structure of the protein and the one or more ligands defines a respective predicted three-dimensional spatial location of each atom in the protein and of each atom in each of the one or more ligands, and
wherein generating the predicted joint 3D structure of the protein and the one or more ligands comprises:
denoising positional data defining estimated positions of each of a plurality of atoms in a complex comprising the protein and the one or more ligands over a sequence of denoising time steps using the generative model and while the generative model is conditioned on the protein-ligand embedding,
wherein the protein-ligand embedding remains constant over the sequence of denoising time steps.

2. The method of claim 1, wherein the generative model comprises a denoising neural network.

3. The method of claim 2, wherein generating, using the generative model and while the generative model is conditioned on the protein-ligand embedding, the predicted joint 3D structure of the protein and the one or more ligands comprises:
generating initial positional data defining a respective initial position of each atom in a complex comprising the protein and the one or more ligands;
wherein the predicted joint 3D structure of the protein and the one or more ligands is defined by the positional data after a final time step in the sequence of denoising time steps.

4. The method of claim 3, wherein generating initial positional data defining the respective initial position of each atom in the complex comprises:
sampling the respective initial position of each atom in the complex from a probability distribution over 3D space.

5. The method of claim 2, wherein denoising the positional data over the sequence of denoising time steps comprises, at each of one or more time steps in the sequence of denoising time steps:
receiving current positional data that defines a respective current position of each atom in the complex at the denoising time step;
generating a denoising output using the denoising neural network and while the denoising neural network is conditioned on the protein-ligand embedding; and
generating positional data that defines a respective position of each atom in the complex at a next time step using the denoising output.

6. The method of claim 5, wherein the denoising output comprises a respective predicted error in the current position of each atom in the complex at the time step.

7. The method of claim 5, wherein generating the denoising output using the denoising neural network and while the denoising neural network is conditioned on the protein-ligand embedding comprises:
generating a set of atom embeddings using an encoder block of the denoising neural network, wherein each atom embedding represents one or more atoms in the complex and is based at least in part on the respective current spatial position of the one or more atoms at the time step;
processing the set of atom embeddings using an update block of the denoising neural network to generate a set of updated atom embeddings; and
processing the set of updated atom embeddings to generate the denoising output.

8. The method of claim 7, wherein the set of atom embeddings includes a respective atom embedding representing each atom included in each of the one or more ligands.

9. The method of claim 7, wherein the set of atom embeddings includes a respective atom embedding representing each atom included in each amino acid of the protein.

10. The method of claim 7, wherein for each amino acid in the protein, the set of atom embeddings includes a respective atom embedding that jointly represents all the atoms included in the amino acid.

11. The method of claim 7, wherein each atom embedding in the set of atom embeddings is based on, for each of the one or more atoms represented by the atom embedding: (i) the current position of the atom at the time step, and (ii) a respective conditioning embedding for the atom that is selected from a collection of embeddings included in the protein-ligand embedding.

12. The method of claim 11, wherein for each atom embedding that represents an atom in the complex that is included in a ligand, the conditioning embedding for the atom comprises an atom-atom embedding corresponding to the atom in the protein-ligand embedding.

13. The method of claim 11, wherein for each atom embedding that represents an atom in the complex that is included in an amino acid of the protein, the conditioning embedding for the atom comprises an amino acid-amino acid embedding corresponding to the amino acid in the protein-ligand embedding.

14. The method of claim 7, wherein the update block of the denoising neural network comprises a sequence of self-attention blocks;
wherein each of the self-attention blocks are configured to apply one or more self-attention operations to a set of current atom embeddings to update the set of current atom embeddings;
wherein each of the one or more self-attention operations are conditioned on the protein-ligand embedding.

15. The method of claim 14, wherein applying a self-attention operation to the set of current atom embeddings to update the set of current atom embeddings comprises:
generating, based on the current set of atom embeddings, a respective intermediate attention score for each pair of current atom embeddings from the set of current atom embeddings;
generating, based on the protein-ligand embedding, a respective attention score bias for each pair of current atom embeddings from the set of current atom embeddings;
generating a respective final attention score for each pair of current atom embeddings from the set of current atom embeddings based on the intermediate attention scores and the attention score biases; and
updating the set of current atom embeddings using the final attention scores.

16. The method of claim 15, wherein for each pair of current atom embeddings from the set of current atom embeddings, generating the final attention score for the pair of current atom embeddings comprises:
summing the intermediate attention score for the pair of current atom embeddings and the attention score bias for the pair of current atom embeddings.

17. The method of claim 15, wherein for each pair of current atom embeddings from the set of current atom embeddings, generating the attention score bias for the pair of current atom embeddings comprises:
processing a respective conditioning embedding selected from a collection of embeddings included in the protein-ligand embedding using a projection neural network to generate the attention score bias.

18. The method of claim 17, wherein for each pair of current atom embeddings that includes: (i) a first atom embedding representing a first atom included in a ligand, and (ii) a second atom embedding representing a second atom included in a ligand, the selected conditioning embedding comprises an atom-atom embedding corresponding to the first atom and the second atom in the protein-ligand embedding.

19. A system comprising:
one or more computers; and
one or more storage devices communicatively coupled to the one or more computers, wherein the one or more storage devices store instructions that, when executed by the one or more computers, cause the one or more computers to perform operations comprising:
obtaining a network input that characterizes a protein and one or more ligands;
processing the network input characterizing the protein and the one or more ligands using an embedding neural network to generate a protein-ligand embedding of the protein and the one or more ligands; and
generating, using a generative model and while the generative model is conditioned on the protein-ligand embedding, a predicted joint three-dimensional (3D) structure of the protein and the one or more ligands,
wherein the predicted joint 3D structure of the protein and the one or more ligands defines a respective predicted three-dimensional spatial location of each atom in the protein and of each atom in each of the one or more ligands, and
wherein generating the predicted joint 3D structure of the protein and the one or more ligands comprises:
denoising positional data defining estimated positions of each of a plurality of atoms in a complex comprising the protein and the one or more ligands over a sequence of denoising time steps using the generative model and while the generative model is conditioned on the protein-ligand embedding,
wherein the protein-ligand embedding remains constant over the sequence of denoising time steps.

20. One or more non-transitory computer storage media storing instructions that when executed by one or more computers cause the one or more computers to perform operations comprising:
obtaining a network input that characterizes a protein and one or more ligands;
processing the network input characterizing the protein and the one or more ligands using an embedding neural network to generate a protein-ligand embedding of the protein and the one or more ligands; and
generating, using a generative model and while the generative model is conditioned on the protein-ligand embedding, a predicted joint three-dimensional (3D) structure of the protein and the one or more ligands,
wherein the predicted joint 3D structure of the protein and the one or more ligands defines a respective predicted three-dimensional spatial location of each atom in the protein and of each atom in each of the one or more ligands, and
wherein generating the predicted joint 3D structure of the protein and the one or more ligands comprises:
denoising positional data defining estimated positions of each of a plurality of atoms in a complex comprising the protein and the one or more ligands over a sequence of denoising time steps using the generative model and while the generative model is conditioned on the protein-ligand embedding,
wherein the protein-ligand embedding remains constant over the sequence of denoising time steps.
